(19) United States
(12) Patent Application Publication
(10) Pub. No.: US 2025/0108051 A1
(43) Pub. Date: Apr. 3, 2025

(54) THERAPEUTIC AGENT FOR CANCER

(71) Applicant: TOHOKU UNIVERSITY, Sendai-shi, Miyagi (JP)

(72) Inventors: Yuya YOSHIDA, Sendai-shi (JP); Masanobu TAKAHASHI, Sendai-shi (JP); Chikashi ISHIOKA, Sendai-shi (JP); Sakura TANIGUCHI, Sendai-shi (JP)

(21) Appl. No.: 18/832,446

(22) PCT Filed: Jan. 17, 2023
(86) PCT No.: PCT/JP2023/001111

§ 371 Date: Jul. 23, 2024

(30) Foreign Application Priority Data
Jan. 27, 2022 (JP) 2022-011064

(51) Int. Cl. A61K 31/506; A61K 31/192; A61K 31/203; A61K 31/4184; A61K 31/519; A61K 39/00; A61P 35/00; C07K 16/22; C07K 16/28

(52) U.S. Cl. CPC A61K 31/506; A61K 31/192; A61K 31/203; A61K 31/4184; A61K 31/519; A61P 35/00; C07K 16/22; C07K 16/2863; A61K 2039/505

(57) ABSTRACT

A therapeutic agent for cancer includes a compound acting on a retinoid receptor, and a BRAF inhibitor. A therapeutic agent for cancer is a therapeutic agent for cancer to be used in combination with a BRAF inhibitor, and contains a compound acting on a retinoid receptor. The compound acting on a retinoid receptor may be a retinoid compound or a derivative thereof. The compound acting on a retinoid receptor may be ATRA, tamibarotene, or bexarotene. The BRAF inhibitor may be dabrafenib or encorafenib. The therapeutic agent for cancer may further contain an MEK inhibitor. The therapeutic agent for cancer may further contain a compound acting on EGF or an EGF receptor.

### CROSS-REFERENCE TO RELATED APPLICATIONS

This application is the U.S. National Stage entry of International Application No. PCT/JP2023/001111, filed on Jan. 17, 2023, which, in turn, claims priority to Japanese Patent Application No. 2022-011064, filed on Jan. 27, 2022, both of which are hereby incorporated herein by reference in their entireties for all purposes.

### TECHNICAL FIELD

The present invention relates to a therapeutic agent for cancer, which contains a component for enhancing an effect of a proliferation inhibitor.

### BACKGROUND ART

In recent years, cancers such as colorectal cancer, lung cancer, breast cancer, and prostate cancer are cancers with a notable increase in incidence, and an effective therapy thereof is an ongoing goal from the basic research to the medical field. Among carcinogenic mechanisms of these cancers, genetic and chromosomal instability is often recognized. Among them, BRAF gene mutation has been reported in thyroid cancer, malignant melanoma, colorectal cancer, ovarian cancer, prostate cancer, and other cancers the like.

RAF family kinases containing BRAF function as important regulatory factors of the MEK-ERK MAP kinase signaling pathway downstream of RAS. This transduction pathway contributes to various cellular activities including cell cycles, proliferation or differentiation of cells, angiogenesis, apoptosis, migration, and metastasis.

An active mutation BRAF V600E, which causes single amino acid substitution in a kinase site, occupies 80% or more of the BRAF gene mutations in the cancers. The BRAF V600E mutation is recognized in thyroid cancer (59%), malignant melanoma (50%), colorectal cancer (10%), and lung cancer (6%). The BRAF V600E-mutated colorectal cancer is known to be inferior in prognosis compared with wild-type or RAS-mutated colorectal cancer.

As a drug therapy for cancers accompanied by gene mutation, a drug therapy using a molecularly-targeted therapeutic drug has been known. Examples of the molecularly-targeted therapeutic drug include bevacizumab, which is monoclonal a antibody against a vascular endothelial growth factor (VEGF), and cetuximab and panitumumab, which are monoclonal antibodies against an epidermal growth factor receptor (EGFR).

As therapy for BRAF V600E-mutated colorectal cancer, therapy using a standard therapy (FOLFIRI or CPT-11, cetuximab) as a secondary therapy has been known. In addition, in the BEACON III test, there is an example showing extension of overall survival by using encorafenib, binimetinib, and cetuximab in combination. Dabrafenib has been known as a BRAF inhibitor, and binimetinib and trametinib have been known as MEK inhibitors of the pathway.

PTL 1 discloses a pharmaceutical composition in which a BRAF inhibitor and an MEK inhibitor are combined. In this technique, the pharmaceutical composition is intended to be used for treatment and inhibition of metastasis of a BRAF-mutated cancer, especially melanoma, and treatment of reducing severity, or reducing or inhibiting risks.

As described above, regarding the BRAF-mutated cancer, a drug therapy technique using various inhibitors and a combination thereof has been developed. However, the prognosis remains poor, and more effective therapeutic methods are required. In particular, further therapeutic effects are strongly required for the colorectal cancer.

The inventors have paid attention to the effect of inhibiting proliferation by the BRAF inhibitor and the MEK inhibitor as these therapeutic methods, and have conducted studies on a method for enhancing the therapeutic effects.

### CITATION LIST

### Patent Literature

PTL 1: JP2016-510748A

### SUMMARY OF INVENTION

### Technical Problem

The invention has been made in view of the above circumstances, and an object thereof is to provide a therapeutic agent for cancer, which contains a component exhibiting a synergistic effect with a BRAF inhibitor, exhibits a strong antitumor effect, exhibits an effect of enhancing a therapeutic effect on cells resistive to a BRAF inhibitor, an anti-EGFR antibody, and an MEK inhibitor, and is particularly effective for therapy for a gene-mutated cancer.

### Solution to Problem

In order to solve the problems, the invention has the following aspects.

A therapeutic agent for cancer according to a first aspect of the invention contains a compound acting on a retinoid receptor, and a BRAF inhibitor.

A therapeutic agent for cancer according to a second aspect of the invention is a therapeutic agent for cancer to be used in combination with a BRAF inhibitor, and contains a compound acting on a retinoid receptor.

The compound acting on a retinoid receptor may be a retinoid compound or a derivative thereof.

The compound acting on a retinoid receptor may be tretinoin, tamibarotene, or bexarotene.

The BRAF inhibitor may be dabrafenib or encorafenib.

The therapeutic agent for cancer according to the first aspect of the invention may further contain an MEK inhibitor.

The MEK inhibitor may be trametinib or binimetinib.

The therapeutic agent for cancer according to the first aspect of the invention may further contain a compound acting on an epidermal growth factor or an epidermal growth factor receptor.

The compound acting on an epidermal growth factor or an epidermal growth factor receptor may be bevacizumab, cetuximab, or panitumumab.

The therapeutic agent for cancer according to the first or second aspect of the invention may be a therapeutic agent for a BRAF-mutated cancer.

The therapeutic agent for cancer according to the first or second aspect of the invention may be a therapeutic agent for a colorectal cancer.

### Advantageous Effects of Invention

According to the therapeutic agent for cancer of the above aspects, it is possible to provide a therapeutic agent for cancer, which contains a component exhibiting a synergistic effect with a BRAF inhibitor, exhibits a strong antitumor effect, exhibits an effect of enhancing a therapeutic effect on cells resistive to a BRAF inhibitor, an anti-EGFR antibody, and an MEK inhibitor, and is particularly effective for therapy for a gene-mutated cancer.

### BRIEF DESCRIPTION OF DRAWINGS

FIG. 1 is a graph showing enhancement in inhibitory effects of tretinoin (TRE or ATRA), dabrafenib (DAB), and trametinib (TRA) in the Examples.

FIG. 2 is a graph showing enhancement of inhibitors by ATRA in various colorectal cancer cell lines in the Examples.

FIG. 3 is a graph showing results obtained by using an RKO cell line regarding an effect of various compounds for acting on a retinoid receptor in the Examples.

FIG. 4 is a graph showing results obtained by using a HT29 cell line regarding an effect of various compounds for acting on a retinoid receptor in the Examples.

FIG. 5 is a graph showing results obtained by using a CO115 cell line regarding an effect of various compounds for acting on a retinoid receptor in the Examples.

FIG. 6 is a graph showing an effect of enhancing an inhibitor by ATRA on an encorafenib/cetuximab resistant strain in the Examples.

FIG. 7 is a graph showing a change in tumor volume caused by in vivo ATRA in the Examples.

FIG. 8 is a graph showing results obtained by using various colorectal cancer cell lines regarding an effect of retinol in the Examples.

FIG. 9 is a graph showing results obtained by using various colorectal cancer cell lines regarding an effect of tamibarotene in the Examples.

FIG. 10 is a graph showing results obtained by using various colorectal cancer cell lines regarding an effect of bexarotene in the Examples.

FIG. 11 is a graph showing results of an Annexin V-propidium iodide iodide (PI) assay using an RKO cell line in the Examples.

FIG. 12 is a graph showing results of an Annexin V-PI assay using a HT29 cell line in the Examples.

FIG. 13 is an image and a graph showing results of analyzing an expression change of p-MEK using a Western blot in the Examples.

FIG. 14 is an image showing results of analyzing expression changes of p-ERK and ERK using a Western blot in the Examples.

FIG. 15 is an image and a graph showing results of analyzing p-AKT using a Western blot in the Examples.

FIG. 16 is an image and a graph showing results of analyzing an expression change of PARP using a Western blot in the Examples.

FIG. 17 is an image showing results of analyzing an expression change of proteins related to the Bcl-2 family using a Western blot in the Examples.

FIG. 18 is a graph in which a signal intensity of each band shown in FIG. 17 is quantified.

FIG. 19 is an image showing results of analyzing protein expression of endogenous RARα and RXRα in each BRAF-mutated colorectal cancer cell line in the Examples using a Western blot.

FIG. 20 is an image and a graph showing results of analyzing expression of RARα and RXRα in RKO cell lines under RARα or RXRα knockdown in the Examples using a Western blot.

FIG. 21 is a graph showing results of studies on proliferation inhibition effects generated by combined use of ATRA, encorafenib, and binimetinib, or combined use of bexarotene, encorafenib, and binimetinib in an RKO cell line under RARα or RXRα knockdown in the Examples.

FIG. 22 is an image and a graph showing results of analyzing expression of RXRα in HT29 cell line under RXRα knockdown in the Examples using a Western blot.

FIG. 23 is a graph showing results of studies on proliferation inhibition effects generated by combined use of TRE, encorafenib (ENC), and binimetinib (BIN), or combined use of bexarotene (BEX), encorafenib (ENC), and binimetinib (BIN) in a HT29 cell line under RXRα knockdown in the Examples.

FIG. 24 is an image and a graph showing results of analyzing expression of cleaved PARP caused by combined use of TRE, ENC, BIN, and cetuximab (CET) in an RKO cell line under RARα or RXRα knockdown in the Examples.

FIG. 25 is a graph showing results of studies on an antitumor effect generated by combined use of TRE and ENC, BIN and CET, or ENC and CET in a subcutaneous transplanted tumor mouse model in the Examples.

FIG. 26 shows immunohistochemical staining images (magnification: 400×), using anti-Ki-67 antibodies, of a subcutaneous transplanted tumor mouse model in the Examples. Note that the scale bar is 100 μm.

FIG. 27 is a graph showing a Ki-67 positive cell proportion (%) calculated using the stained image in FIG. 26.

### DESCRIPTION OF EMBODIMENTS

Hereinafter, a therapeutic agent for cancer according to the invention will be described by showing embodiments. However, the invention is not limited to the following embodiments.

### (Therapeutic Agent for Cancer)

The therapeutic agent for cancer according to the present embodiment contains a compound acting on a retinoid receptor, and a BRAF inhibitor.

### (Compound Acting on Retinoid Receptor)

Examples of the compound acting on a retinoid receptor include a retinoid compound and a derivative thereof. The retinoid compound widely refers to vitamin A-derived compounds, derivatives of vitamin A, or analogues of vitamin A. Specific examples of the retinoid compound include retinoic acid or a derivative thereof.

Examples of the compound acting on a retinoid receptor include retinol, tretinoin (ATRA, all-trans retinoic acid), isotretinoin (13-cis-retinoic acid), and alitretinoin (9-cis-retinoic acid), as a first-generation retinoid.

Examples of a second-generation retinoid include etretinate and acitretin.

Examples of a third-generation retinoid include adapalene, bexarotene, tazarotene, and tamibarotene.

Examples of a fourth-generation retinoid include trifarotene.

The first-generation and the second-generation retinoids each have flexibility when a single bond and a double bond appear alternately, and therefore, the first-generation and the second-generation retinoids each can bind to one or more retinoid receptors.

Among the above-described retinoids, adapalene is a selective RAR agonist, bexarotene is a selective RXR agonist, and tamibarotene is an agonist of RAR/RXR. The selectivity of RAR A is high. Trifarotene is a selective RARγ agonist.

In the embodiment, as the compound acting on a retinoid receptor, it is more preferable to use retinol, ATRA, tamibarotene, or bexarotene, among them, and it is still e preferable to use ATRA, tamibarotene, or bexarotene.

These compounds for acting on the retinoid receptor can enhance the effect of the BRAF inhibitor. That is, the compound acting on a retinoid receptor can also be referred to as an antitumor effect-enhancing agent for the BRAF inhibitor. In addition, a synergistic effect of further enhancing the effect is exhibited by using these compounds for acting on the retinoid receptor in combination with the BRAF inhibitor and the MEK inhibitor. That is, the compound acting on a retinoid receptor can also be referred to as an antitumor effect-enhancing agent for the BRAF inhibitor and the MEK inhibitor.

### (Inhibitor)

The therapeutic agent for cancer according to the embodiment contains a BRAF inhibitor. The expression “the therapeutic agent for cancer contains a BRAF inhibitor” includes a case where the therapeutic agent for cancer is provided in a form in which another component and a component containing the BRAF inhibitor are separately stored and used in combination, in addition to a form in which the BRAF inhibitor is contained in the same formulation.

BRAF refers to a gene that expresses a B-Raf protein. The BRAF inhibitor is a component widely containing a component that inhibits expression of a B-Raf protein, and more specifically, a component known to inhibit a BRAF gene. As the BRAF inhibitors, those commonly known can be used, and those used as components for cancer therapy are preferable.

In the therapeutic agent for cancer according to the embodiment, it is more preferable to use dabrafenib or encorafenib as the BRAF inhibitors.

As a combination of the above-described compounds for acting on the retinoid receptor and the BRAF inhibitor, it is more preferable to use ATRA and dabrafenib, ATRA and encorafenib, tamibarotene and dabrafenib, tamibarotene and encorafenib, bexarotene and dabrafenib, or bexarotene and encorafenib.

The therapeutic agent for cancer according to the embodiment can further contain another component depending on the type of cancer, in addition to the BRAF inhibitor and the compound acting on the retinoid receptor.

The therapeutic agent for cancer according to the embodiment preferably further contains an MEK inhibitor. MEK refers to a kinase enzyme MEK1 or MEK2 of a mitogen-activated protein kinase, and is a kinase enzyme for phosphating the mitogen-activated protein kinase. The MEK inhibitor refers to components widely containing a component that inhibits the expression of an MEK protein (enzyme), and more specifically, a component known to inhibit an MEK gene. As the MEK inhibitor, those commonly known can be used, and those used as components for cancer therapy are preferable.

In the therapeutic agent for cancer according to the embodiment, it is more preferable to use trametinib or binimetinib as the MEK inhibitor.

As a combination of the above-described compounds for acting on a retinoid receptor, the BRAF inhibitor, and the MEK inhibitor, it is more preferable to use a combination of ATRA, encorafenib, and binimetinib, a combination of tamibarotene, encorafenib, and binimetinib, or a combination of bexarotene, encorafenib, and binimetinib. Alternatively, it is more preferable to use a combination of ATRA, dabrafenib, and trametinib, a combination of tamibarotene, dabrafenib, and trametinib, or a combination of bexarotene, dabrafenib, and trametinib.

It is known that it is effective to use the MEK inhibitor in combination with a BRAF inhibitor according to the related art in treatment of cancer. Further, the compound acting on a retinoid receptor according to the embodiment also enhances the effect of the MEK inhibitor in addition to the BRAF inhibitor. Therefore, a further synergistic effect can be obtained by using a compound acting on a retinoid receptor, a BRAF inhibitor, and an MEK inhibitor in combination in the therapeutic agent for cancer.

The therapeutic agent for cancer according to the embodiment may contain another component known to be used in combination with a BRAF inhibitor or an MEK inhibitor in treatment of cancer.

For example, a compound acting on an epidermal growth factor (EGF) or an epidermal growth factor receptor (EGFR) may be contained. As such a compound, an antibody against VEGF or an antibody against EGFR may be used. More specifically, bevacizumab, cetuximab, panitumumab, or the like may be contained.

As a combination of the above-described compounds for acting on a retinoid receptor, the BRAF inhibitor, and the compounds for acting on EGF or EGFR, it is preferable to use a combination of ATRA, encorafenib, and cetuximab, a combination of tamibarotene, encorafenib, and cetuximab, or a combination of bexarotene, encorafenib, and cetuximab. Alternatively, it is preferable to use a combination of ATRA, dabrafenib, and cetuximab, a combination of tamibarotene, dabrafenib, and cetuximab, or a combination of bexarotene, dabrafenib, and cetuximab. Alternatively, it is preferable to use a combination of ATRA, dabrafenib, and panitumumab, a combination of tamibarotene, dabrafenib, and panitumumab, or a combination of bexarotene, dabrafenib, and panitumumab.

As a combination of the above-described compounds for acting on the retinoid receptor, the BRAF inhibitor, the MEK inhibitor, and the compounds for acting on EGF or EGFR, it is preferable to use a combination of ATRA, encorafenib, binimetinib, and cetuximab, a combination of tamibarotene, encorafenib, binimetinib, and cetuximab, or a combination of bexarotene, encorafenib, binimetinib, and cetuximab. Alternatively, it is preferable to use a combination of ATRA, dabrafenib, trametinib, and cetuximab, a combination of tamibarotene, dabrafenib, trametinib, and cetuximab, or a combination of bexarotene, dabrafenib, trametinib, and cetuximab.

### (Use of Therapeutic Agent for Cancer)

The therapeutic agent for cancer according to the embodiment can be widely used for pharmaceuticals, pharmaceutical compositions, anticancer agents, anticancer compositions, therapeutic drugs for cancer, and the like. These therapeutic agents for cancer can be used for therapy, prevention, and treatment associated therewith of cancers.

The therapeutic agent for cancer according to the embodiment can be preferably used for the therapy of mutated cancers, that is, cancers caused by mutations of genes.

The therapeutic agent for cancer according to the embodiment can be preferably used for the therapy of a BRAF-mutated cancer among these mutated cancers. Among the BRAF-mutated cancers, it can be used for BRAF V600E-mutated cancers, and in particular, for BRAF V600E-mutated colorectal cancer. Here, the BRAF V600E-mutated cancer refers to a cancer that exhibits a positive reaction in a BRAF V600 mutation test.

The therapeutic agent for cancer according to the embodiment can also be used for the therapy of various cancers. In the embodiment, the term “cancer” refers to a physiological state mainly characterized by disordered cell proliferation, widely refers to a malignant tumor (cancer), and is also referred to as the term “cancerous” or “malignant”. Examples of the cancer include carcinoma, lymphoma, leukemia, blastoma, and sarcoma. More specific examples of the cancer include squamous cell carcinoma, myeloma, small cell lung cancer, non-small cell lung cancer, glioma, Hodgkin lymphoma, Non-Hodgkin lymphoma, acute myeloid leukemia, multiple myeloma, gastrointestinal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma, cervical cancer, brain cancer, stomach cancer, bladder cancer, hepatoma, breast cancer, colon cancer, and head and neck cancers. Among them, the therapeutic agent for cancer according to the embodiment can be used for, for example, a cancer in a digestive organ or lung cancer. Among them, the therapeutic agent for cancer according to the embodiment can be particularly preferably used for the therapy of colorectal cancer.

The therapy of cancer widely includes ameliorations in symptoms such as a decrease in the number of cancer cells, a decrease in the tumor size, a decrease in the rate of cancer cell invasion into peripheral organs, and a decrease in metastasis of tumor and the proliferation rate of tumor.

The therapeutic agent for cancer according to the embodiment can be used in therapeutic methods for the above-described cancers, specifically, methods of treating, inhibiting, reducing the severity of, reducing the risk of, or inhibiting the above-described cancers, or treating metastasis of cancers.

The therapeutic agent for cancer according to the embodiment can be used for the production of other agents or compositions used for the therapy of the above-described cancers.

### (Effect of Therapeutic Agent for Cancer)

The therapeutic agent for cancer according to the embodiment contains a component exhibiting a synergistic effect with the BRAF inhibitor, and exhibits a strong antitumor effect. Specifically, the effect of enhancing the proliferation inhibition effect due to the BRAF inhibitor by 20% or more is achieved. These effects were confirmed to inhibit proliferation in a plurality of BRAF-mutated colorectal cancer cell lines.

It is predicted that these reaction mechanisms are related to the effects of acting on RXR, RAR, RFR, or the like in the compounds for acting on the retinoid receptor. Therefore, a compound which is a selective agonist for RXR, RAR, RFR, and the like may be effective.

The therapeutic agent for cancer according to the embodiment also exhibits a synergistic effect with the effect of an MEK inhibitor. That is, a higher cancer therapeutic effect is exhibited by further containing the MEK inhibitor.

The inventors have found that a compound acting on a retinoid receptor enhances the effect of a BRAF inhibitor. In addition, the inventors have found that a synergistic effect of further enhancing the effect is exhibited by using the compound acting on a retinoid receptor in combination with a BRAF inhibitor and an MEK inhibitor.

That is, the inventors have focused on the possibility of obtaining a higher cancer therapeutic effect by using a compound capable of enhancing the effects of the BRAF inhibitor and the MEK inhibitor in combination in the therapeutic agent for cancer. Then, screening has been performed to obtain a component capable of enhancing the effect of the inhibitor, and the configuration of the embodiment has been obtained.

The compound acting on a retinoid receptor has been mainly used in the dermatology field in the related art and has not been noted in the tumor field. In addition, a synergistic effect with a BRAF inhibitor and a synergistic effect with other components used for cancer treatment such as an MEK inhibitor have not been known.

The therapeutic agent for cancer according to the embodiment exhibits an effect of enhancing therapeutic effects on cells having resistance to BRAF inhibitors, compounds for acting on EGF or EGFR, and preferably anti-EGFR antibodies and MEK inhibitors.

That is, there is a possibility that the therapeutic agent for cancer according to the embodiment is also effective for a cell tissue that has obtained resistance to BRAF inhibitors, compounds for acting on EGF or EGFR, preferably anti-EGFR antibodies, MEK inhibitors, or other components. In addition, there is a possibility that a therapeutic effect can be exerted on a patient for whom a therapeutic agent containing these components is no longer effective.

Due to these effects, the therapeutic agent for cancer according to the embodiment is particularly effective for the therapy of gene-mutated cancers.

In the therapeutic agent for cancer according to the embodiment, an administration amount of the compound acting on a retinoid receptor can be an administration amount that has been used clinically in the related art. Alternatively, in the therapeutic agent for cancer according to the embodiment, the above-described enhancing effect can be exhibited even when the administration amount of the compound acting on a retinoid receptor is about ½, ⅓, ¼, ⅕, ⅙, 1/7, ⅛, or 1/9 of the administration amount used clinically in the related art.

The administration amount of each of the BRAF inhibitor, the MEK inhibitor, and the compounds for acting on EGF or EGFR can also be an administration amount that has been used clinically in the related art. Alternatively, the therapeutic effect of these drugs is enhanced by using the drugs in combination with the compound acting on a retinoid receptor, and therefore, it is possible to reduce the administration amount to about ½, ⅓, ¼, ⅕, ⅙, 1/7, ⅛, or 1/9 of the usage amount as compared with the administration amount used clinically in the related art. In addition, the drug is used in combination with the compound acting on a retinoid receptor, and the drug is administered according to an administration amount that has been used clinically in the related art, so that the administration amount of the drug may be reduced when a certain level of therapeutic effect is observed. By reducing the administration amount, the toxicity of the drug can be reduced, and the burden on the patient to be administered can be reduced.

(Therapeutic Agent for Cancer to be Used in Combination with BRAF Inhibitor)

A therapeutic agent for cancer in another embodiment relative to the embodiment described above is a therapeutic agent for cancer to be used in combination with a BRAF inhibitor, and contains a compound acting on a retinoid receptor.

The therapeutic agent for cancer according to the embodiment contains at least a compound acting on a retinoid receptor. The therapeutic agent for cancer according to the embodiment is used in combination with a BRAF inhibitor.

Here, the expression “the therapeutic agent for cancer is used in combination with the BRAF inhibitor” widely includes aspects in which the therapeutic agent for cancer and the BRAF inhibitor are used in combination. For example, an aspect in which the therapeutic agent for cancer according to the embodiment and the BRAF inhibitor are administered at the same time and used is included. The expression “used by being administered at the same time” includes a case where the therapeutic agent for cancer and the BRAF inhibitor are formed in the same formulation, for example, a case where the two are administered as a combination drug, and also includes a case where the two are formed in separated formulations and are administered at the same time. In addition, the term “used in combination” also includes a case where the two are used not at the same time but sequentially. The term “sequentially used” means that the therapeutic agent for cancer and another component are continuously used. The number of administrations and the administration amounts of the therapeutic agent for cancer and another component may be the same as or different from each other. The form of administration may be oral administration, injection, or the like.

During the administration, each can be orally administrated every day, and in this case, the administration time may be substantially the same on the same day or may be separated times. More specifically, for example, the compound acting on a retinoid receptor and the BRAF inhibitor may be orally administrated every day, and the compound acting on EGF or EGFR, preferably an anti-EGFR antibody, may be administered once a week or every other week by intravenous injection.

As the BRAF inhibitor, the same components as those in the embodiment described above can be used.

The therapeutic agent for cancer according to the embodiment may be used in combination with the embodiment described above and an MEK inhibitor, a compound acting on EGF or EGFR, preferably an anti-EGFR antibody.

In addition, the therapeutic agent for cancer according to the embodiment may contain another component according to the above-described embodiment, or may be used in combination therewith.

### OTHER EMBODIMENTS

Still another embodiment relative to the embodiment described above includes a therapeutic agent for cancer, in which a BRAF inhibitor is used in combination with a retinoid receptor. Still another embodiment includes a method for ameliorating a symptom of cancer by using a BRAF inhibitor and a retinoid receptor in combination, and a method for treating or preventing cancer by using a BRAF inhibitor and a retinoid receptor in combination.

A therapeutic agent for cancer according to still another embodiment is a therapeutic agent for cancer to be used in combination with a compound acting on a retinoid receptor, and contains a BRAF inhibitor. A therapeutic agent for cancer according to still another embodiment is a therapeutic agent for cancer to be used in combination with a compound acting on a retinoid receptor, and contains an MEK inhibitor.

Examples of other embodiments of the invention include the following.
a) A method for ameliorating a symptom of cancer in a subject, the method including administering a compound acting on a retinoid receptor and a BRAF inhibitor to the subject.
b) A method for ameliorating a symptom of cancer in a subject, the method including administering a compound acting on a retinoid receptor and a BRAF inhibitor to the subject, in which the subject has a BRAF-mutated cancer.
c) A method for ameliorating a symptom of cancer in a subject, the method including administering a compound acting on a retinoid receptor and a BRAF inhibitor to the subject, in which the subject has colorectal cancer.
d) A method for treating or preventing cancer in a subject, the method including administering a compound acting on a retinoid receptor and a BRAF inhibitor to the subject.
e) A method for inhibiting the progression of cancer in a subject, the method including administering a compound acting on a retinoid receptor and a BRAF inhibitor to the subject.
f) A compound acting on a retinoid receptor for use in combination with a BRAF inhibitor to ameliorate a symptom of cancer.
g) A compound acting on a retinoid receptor for use in combination with a BRAF inhibitor to ameliorate a symptom of a BRAF-mutated cancer.
h) A compound acting on a retinoid receptor for use in combination with a BRAF inhibitor to ameliorate a symptom of colorectal cancer.
i) A compound acting on a retinoid receptor for use in combination with a BRAF inhibitor to treat or prevent cancer.
j) A compound acting on a retinoid receptor for use in combination with a BRAF inhibitor to inhibit the progression of cancer.
k) A pharmaceutical composition containing a compound acting on a retinoid receptor for use in combination with a BRAF inhibitor to improve a symptom of cancer.
l) A pharmaceutical composition containing a compound acting on a retinoid receptor for use in combination with a BRAF inhibitor to improve a symptom of a BRAF-mutated cancer.
m) A pharmaceutical composition containing a compound acting on a retinoid receptor for use in combination with a BRAF inhibitor to ameliorate a symptom of colorectal cancer.
n) A pharmaceutical composition containing a compound acting on a retinoid receptor for use in combination with a BRAF inhibitor to treat or prevent cancer.
o) A pharmaceutical composition containing a compound acting on a retinoid receptor for use in combination with a BRAF inhibitor to inhibit the progression of cancer.
p) Use of a compound acting on a retinoid receptor in combination with a BRAF inhibitor to produce a pharmaceutical composition for ameliorating a symptom of cancer.
q) Use of a compound acting on a retinoid receptor in combination with a BRAF inhibitor to produce a pharmaceutical composition for ameliorating a symptom of a BRAF-mutated cancer.
r) Use of a compound acting on a retinoid receptor in combination with a BRAF inhibitor to produce a pharmaceutical composition for ameliorating a symptom of colorectal cancer.
s) Use of a compound acting on a retinoid receptor in combination with a BRAF inhibitor to produce a pharmaceutical composition for treating or preventing cancer.
t) Use of a compound acting on a retinoid receptor in combination with a BRAF inhibitor to produce a pharmaceutical composition for inhibiting the progression of cancer.
u) A therapeutic agent kit for cancer, the kit including a BRAF inhibitor and a compound acting on a retinoid receptor.
v) A therapeutic agent kit for cancer, the kit including a BRAF inhibitor, a compound acting on a retinoid receptor, and an MEK inhibitor.
w) A therapeutic agent kit for cancer, the kit including a BRAF inhibitor, a compound acting on a retinoid receptor, and an MEK inhibitor or a compound acting on EGF or EGFR.

### EXAMPLES

Hereinafter, the effects of the invention will be made more apparent from Examples and Comparative Examples. It should be noted that the invention is not limited to the following Examples, and can be appropriately modified without departing from the scope of the invention.

### (Used Drug)

Dabrafenib (catalog #D-5699) was purchased from LC Laboratories (Woburn, MA, USA), Trametinib (catalog #16292) and ATRA (all-trans Retinoic Acid) (catalog #11017) were purchased from Cayman Chemical (Ann Arbor, MI, USA).

Encorafenib (catalog #16994, HY-15605), Binimetinib (catalog #16996, HY-15202) were purchased from Cayman Chemical (Ann Arbor, MI, USA) and MedChemoExpress (Monmouth Junction, NJ, USA).

Retinol (catalog #S5592) and Tamibarotene (catalog #S4260) were purchased from Selleck Chemicals (Houston, TX, USA).

Bexarotene (catalog #HY-14171) was purchased from MedChemoExpress (Monmouth Junction, NJ, USA).

Cetuximab (Erbitax) was purchased from Merck Serono (Tokyo, Japan).

### (Colorectal Cancer Cell Line)

For four kinds of colorectal cancer cell lines used in the tests described below, RKO cells were purchased in 2015 from American Type Culture Collection (Manassas, VA, USA).

Regarding WiDR cells, cells assigned by JCRB cell bank (Osaka, Japan) of National Institutes of Biomedical Innovation, Health and Nutrition were used.

Regarding HT29, CO115, LIM2405, and COLO205, those obtained from Dr. John M. Mariadason (Ludwig Institute for Cancer Research, Melbourne, Vic, Australia) were used.

The RKO cells, WiDR cells, HT29 cells, and CO115 cells were cultured in Dulbecco's Modified Eagle's Medium (Sigma-Aldrich Inc., St. Lois, MO, USA) containing 10% fine bovine serum (fetal bovine serum, FBS) under the conditions of 37° C. and a CO₂ concentration of 5%.

### Test Example 1: Search for Compounds that Enhance Sensitivity of BRAF Inhibitor and MEK Inhibitor

### Test Method

Using SCAD Inhibitor Kit4 containing 80 compounds, compounds which have a possibility of enhancing the antitumor effect of a BRAF inhibitor and an MEK inhibitor in RKO cells, were screened.

As the SCADS Inhibitor Kit, one provided by the Molecular Profiling Committee of the Grants-in-Aid for Scientific Research “Advanced Animal Model Support (AdAMS)” in the innovation field of the Ministry of Education, Culture, Sports, Science and Technology was used.

The RKO cells were seeded on a 96-well plate with the number of cells of 2.7×10³ cells/well, and 24 hours later, SCADS Inhibitor Kit 4 ver 2.3 was administered at 500 nM. Two plates treated in the same manner were prepared, and 50 nM of the BRAF inhibitor Dabrafenib and 5 nM of the MEK inhibitor Trametinib were administered to one of the plates. After incubation at 37° C. for 72 hours after the drug administration, cell viability was measured by an MTT assay (described below). The cell viability was evaluated for RKO cells administered with only DMSO.

The MTT (Thiazolyl blue tetrazolium bromide) assay was performed as follows.

RKO cells were seeded on a 96-well plate with the number of cells of 1.5×10³ cells/well, CO115 cells were seeded on a 96-well plate with the number of cells of 2.5×10³ cells/well, and HT29 cells and WiDR cells were respectively seeded on 96-well plates with the number of cells of 6×10³ cells/well. After incubation for about 24 hours until the cell density reached 40% to 60%, the drug was administered. After incubation at 37° C. for 72 hours, the MTT assay was performed using Cell Counting Kit-8 (Dojindo Laboratories, Kumamoto, Japan). To each well was added 10 μl of Cell Counting Kit-8 solution, the plates were placed in a culture vessel at 5% CO₂ and 37° C. for 120 minutes to perform a color reaction, and then the absorbance at 450 nm was measured and evaluated with a microplate reader SpectraMax M2e (Molecular Devices, Sunnyvale, CA, USA). Cells administered with 0.1% DMSO were used as a control. The above was performed three or more times as independent experiments.

The synergistic effect was evaluated using Combination Index. For the calculation of Combination Index, CompuSyn software (ComboSyn Inc, NJ, USA) was used. Based on the previous reports, CI<0.7 was defined as a synergistic effect, 0.7≤CI≤−1.0 was defined as a slight synergistic effect/additive effect, and CI>1.0 was defined as antagonism.

### Test Results

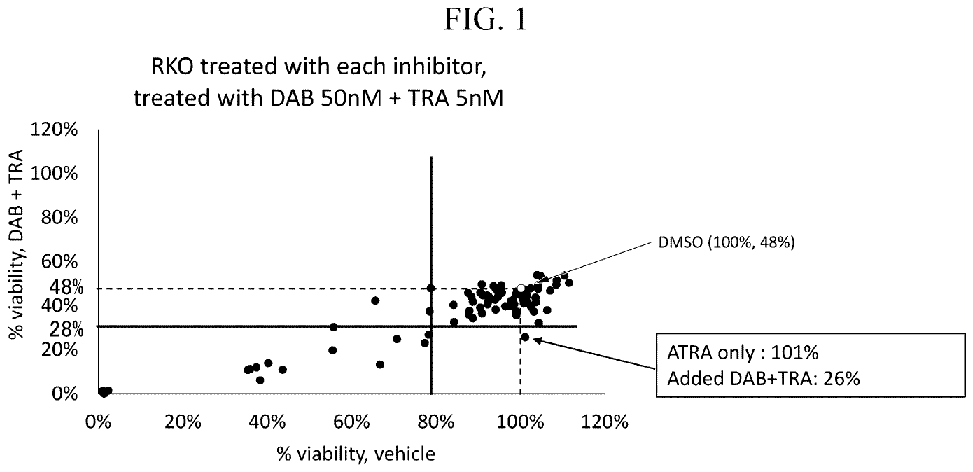

The results of a synergistic effect evaluation in the case of treatment with 0.5 μM of a standard inhibitor kit for 72 hours are shown in FIG. 1. DAB+TRA indicates a value obtained by using 50 nM of dabrafenib (DAB) and 5 nM of trametinib (TRA) in combination. The arrow indicates ATRA. The viability in the case of the ATRA single agent is 101%, and the viability in the case of using a combination of DAB+TRA and ATRA is 26%. The viability in the case of using the combination is significantly lower than 48% in the case of only DAB+TRA (only DMSO as a solvent), that is, the effect of decreasing the viability and the inhibition effect are high. Therefore, ATRA was confirmed to be a compound exhibiting a synergistic effect particularly when used in combination with DAB+TRA.

In the case of using ATRA alone, the proliferation inhibition was less than 20%, and in the case of using a combination of DAB+TRA+ATRA, the inhibition effect was confirmed to be 20% or more higher than the proliferation inhibition caused by DAB+TRA. Therefore, it was shown that ATRA exhibited a synergistic effect with DAB and TRA, and ATRA was a component that enhances the inhibition effect of these components.

### Test Example 2: Effect of ATRA in Each Colorectal Cancer Cell Line

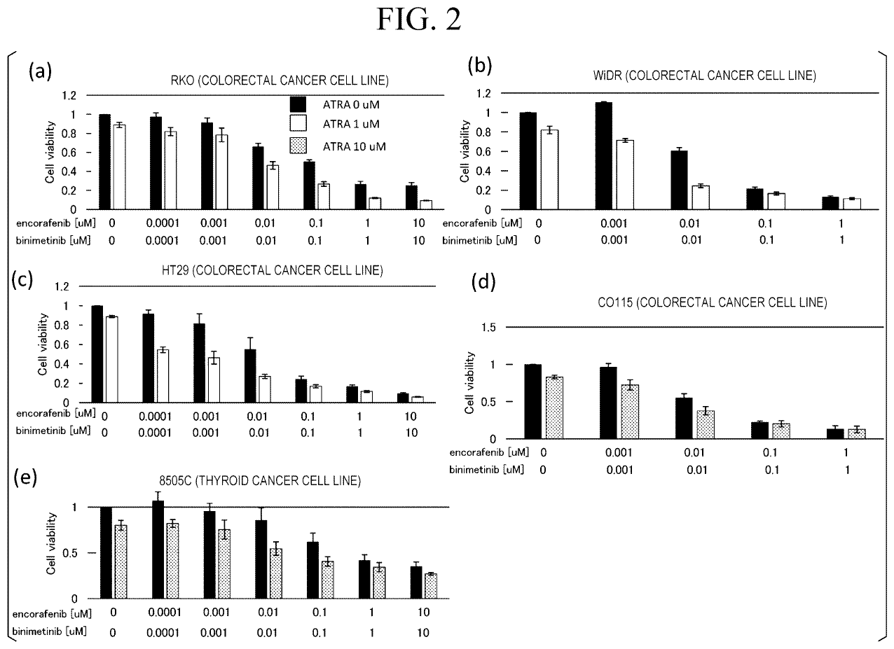

In various kinds of colorectal cancer cell lines, the inhibition effect of a BRAF inhibitor (encorafenib) and an MEK inhibitor (binimetinib) for the viability of cells and the enhancement by ATRA were examined. FIG. 2 shows the viability of colorectal cancer cell lines: RKO in (a), WiDR in (b), HT29 in (c), CO115 in (d), and 8505C in (e). The horizontal axis indicates component concentrations of encorafenib and binimetinib in a range of 0 μM to 10 μM, and the vertical axis indicates the cell viability when 0 μM, 1.0 μM, or 10 μM (each bar graph) of ATRA was administered. As shown in the drawing, encorafenib and binimetinib were used in an equal molar concentration.

Regarding each cell line, IC50 (50% inhibition concentration) was as follows.
RKO (BRAF V600E-mutated cancer colorectal cancer cell line)
ATRA 0 μM: 103
ATRA 1 μM: 7.5
HT29 (BRAF V600E-mutated colorectal cancer cell line)
ATRA 0 μM: 14.5
ATRA 1 μM: 0.32
WIDR (BRAF V600E-mutated colorectal cancer cell line)
ATRA 0 μM: 19.1
ATRA 1 μM: 3.6
CO115 (BRAF V600E-mutated colorectal cancer cell line)
ATRA 0 μM: 14.3
ATRA 10 μM: 4.4
8505C (BRAF V600E-mutated thyroid cancer cell line)
ATRA 0 μM: 398
ATRA 10 μM: 22.8

As shown in each of the drawings, in any of the cell lines, the cell viability decreases and the proliferation of cancer cells is inhibited as the concentrations of encorafenib and binimetinib increase. In all cases, the higher the concentration of ATRA is, the lower the cell viability is. Therefore, it was shown that ATRA increased the cell proliferation inhibition effect of encorafenib and binimetinib in any colorectal cancer cell line.

From these results, it was suggested that therapy of combining ATRA, encorafenib, and binimetinib could exhibit a synergistic effect in BRAF V600E-mutated colorectal cancer cell lines RKO, HT29, WiDR, and CO115.

### Test Example 3: Verification of Cell Proliferation Inhibition Effect of Each Retinoid Compound

The cell proliferation inhibition effect, that is, the effect of enhancing the cell proliferation inhibition action of the BRAF inhibitor and the MEK inhibitor was examined with respect to compounds for acting on a retinoid receptor other than ATRA (tretinoin). As the compounds for acting and on a retinoid receptor, retinol, tamibarotene, bexarotene among the retinoids were used. As the cell lines, the above-described cell lines including RKO, HT29, and CO115 were used. As the BRAF inhibitor and the MEK inhibitor to be used in combination, a component (EB) obtained by mixing encorafenib and binimetinib in equal amounts was used as in Test Example 2.

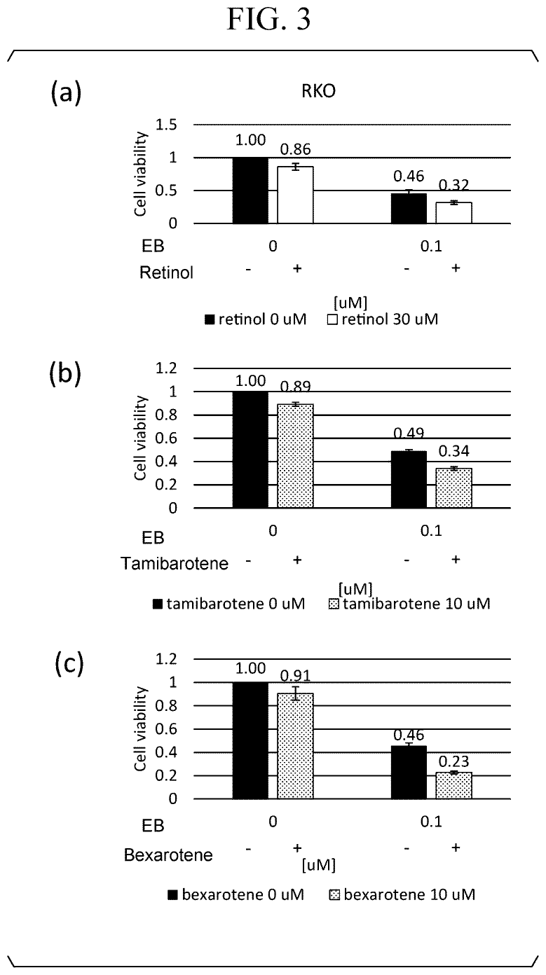

FIG. 3 shows results obtained by using the RKO cell line. (a) of FIG. 3 shows a cell viability in the case of 0 μM or 0.1 μM of EB using retinol (0 μM or 30 μM), (b) of FIG. 3 shows a cell viability in the case of 0 μM or 0.1 μM of EB using tamibarotene (0 μM or 10 μM), and (c) of FIG. 3 shows a cell viability in the case of 0 μM or 0.1 μM of EB using bexarotene (0 μM or 10 μM). As shown in these drawings, in the RKO cell line, with any retinoid among retinol, tamibarotene, and bexarotene, the cell viability was greatly reduced and the effect of enhancing the cell proliferation inhibition action of EB was exhibited in the case of further adding a retinoid to EB.

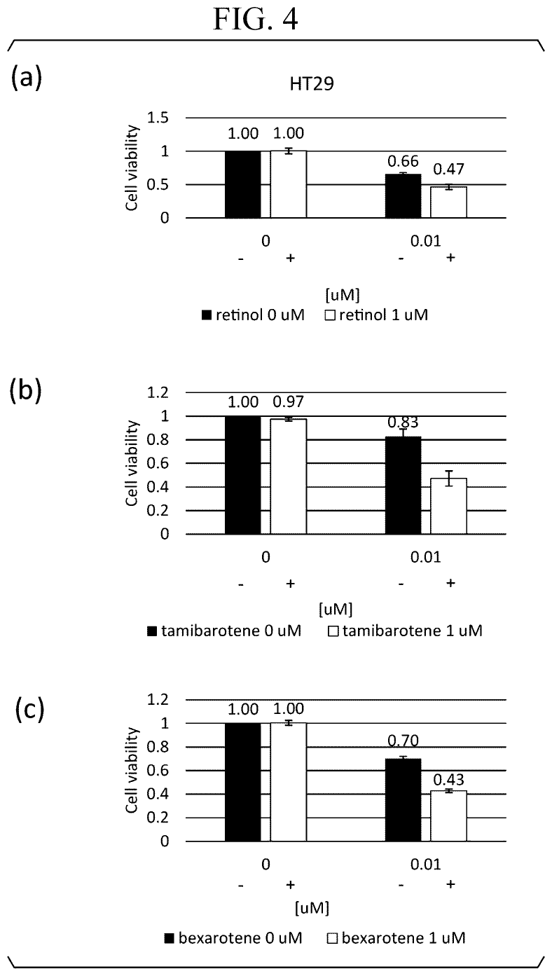

FIG. 4 shows results obtained using the HT29 cell line. (a) of FIG. 4 shows cell viability in the case of 0 μM or 0.01 μM of EB using retinol (0 μM or 1 μM), (b) of FIG. 4 shows cell viability in the case of 0 μM or 0.01 μM of EB using tamibarotene (0 μM or 1 μM), and (c) of FIG. 4 shows cell viability in the case of 0 μM or 0.01 μM of EB using bexarotene (0 μM or 1 μM). As shown in these drawings, in the HT29 cell line, with any retinoid among retinol, tamibarotene, and bexarotene, the cell viability was greatly reduced and the effect of enhancing the cell proliferation inhibition action of EB was exhibited in the case of further adding a retinoid to EB.

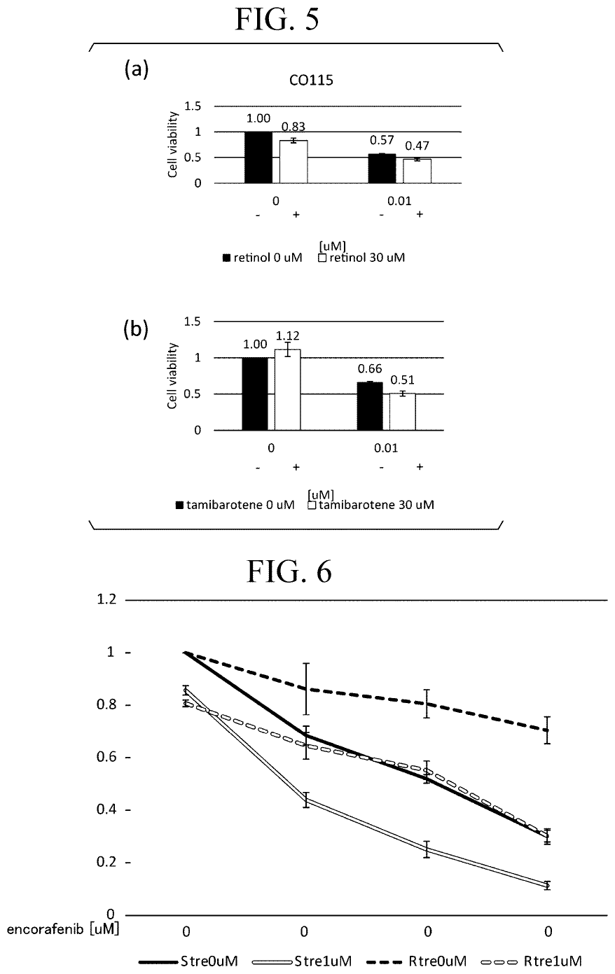

FIG. 5 shows results obtained by using the CO115 cell line. (a) of FIG. 5 shows cell viability in the case of 0 μM or 0.1 μM of EB using retinol (0 μM or 30 μM), and (b) of FIG. 5 shows cell viability in the case of 0 μM or 0.1 μM of EB using tamibarotene (0 μM or 30 μM). As shown in these drawings, in the CO115 cell line, with any retinoid of retinol and tamibarotene, the cell viability was greatly reduced and the effect of enhancing the cell proliferation inhibition action of EB was exhibited in the case of further adding a retinoid to EB.

### Test Example 4: Verification of Effects in Encorafenib/Cetuximab Resistant Strains

Encorafenib/cetuximab resistant strains were constructed, and the inhibitor enhancing effect of ATRA was examined for the resistant strains.

For the construction of the resistant strains, the above-described RKO cells were seeded in 15 mm dishes and a group in which the BRAF inhibitor Encorafenib and an anti-EGFR antibody Cetuximab were administered and a group in which the BRAF inhibitor Encorafenib, the MEK inhibitor Binimetinib, and the anti-EGFR antibody Cetuximab were administered were prepared.

In the RKO cells, the administration was started from Encorafenib of 10 nM, Binimetinib of 10 nM, and Cetuximab of 1 μg/ml, and the amounts of the drugs were gradually increased in consideration of the proliferation rate and administration period to obtain resistant strains.

FIG. 6 shows results of comparing the effects of administration of 0 μM or 1 μM of tretinoin (ATRA) using the resistant strain (R in the drawing) and a sensitive strain (S in the drawing) as a control. The vertical axis represents the viability (ratio), and the horizontal axis represents the administration amount of encorafenib.

From the results shown in the drawing, even when ATRA was not administered (0 μM) to the sensitive strain(S), the viability thereof was decreased in accordance with the administration amount of encorafenib of 0 to 10 μM. In the case where ATRA was administered (1 μM), the viability was further reduced to around 0.1.

The resistant strain (R), to which ATRA was not administered (0 μM), has obtained encorafenib resistance, and thus has a small decrease in the viability with respect to the administration amount of encorafenib, and exhibits a viability of 0.7 to 0.8 even in the case of encorafenib of 10 μm.

However, when ATRA was administered to the resistant strain (R) (1 μM), the viability was reduced to the same level as that when ATRA was not administered to the sensitive strain (a viability around 0.3). From these results, it was shown that ATRA can impart inhibition generated by encorafenib, that is, a cell proliferation inhibition effect, to a cell line that has obtained encorafenib resistance.

Further, regarding each cell line, IC50 (50% inhibition concentration) was as follows.
Sensitive Strain
ATRA 0 μM: 1.23
ATRA 1 μM: 0.0046
Resistant Strain
ATRA 0 μM: >10
ATRA 1 μM: 1.58

From these results, it was revealed that ATRA exhibited the enhancement of effects of BRAF inhibitor+anti-EGFR antibody+−MEK inhibitor even in a cell line (secondary) which was resistant to the BRAF inhibitor of the BRAF-mutated colorectal cancer cell line.

### Test Example 5: Verification of Effect in PDX Model

As an in vivo test, the therapeutic agent for cancer according to the embodiment was administered to a mouse, and the antitumor effect was verified based on a change in tumor size (volume).

Xenograft mouse model was prepared as follows.

Female nude mice (BALB/c-nu) were purchased from Charles River Laboratories Japan (Yokohama, Japan) and raised in a specific pathogen-free environment.

The above-described HT29 cells cultured were collected with trypsin, and suspended in a mixed solution of a culture medium and a Corning Matrigel basement membrane matrix (Corning, NY, USA) so as to reach 1×10⁷ cells/ml.

Into a left abdominal region of each mouse was subcutaneously implanted 0.1 mL of cell suspension. At a time point when a tumor volume reached 150 mm³ to 200 mm³, the mice were randomly divided into a control group, an all-trans Retinoic Acid (ATRA) administration group, an Encorafenib/Cetuximab administration group, and an ATRA/Encorafenib/Cetuximab administration group. Encorafenib was orally administered at a dose of 10 mg/kg daily, and ATRA was orally administered at a dose of 10 mg/kg daily for 21 days. Feeding needles for mice were used for oral administration. Cetuximab was intraperitoneally administered twice a week at a dose of 20 mg/kg. Each solvent was used as the control. The tumor size and the body weight were measured every three days after the start of the therapy, and the volume was estimated using a calculation formula (tumor volume (mm³)=½× major axis (mm)×minor axis (mm)). The animal experiment was approved by the Institutional Animal Care and Use Committee of the Tohoku University, and was performed according to the facility guideline of the Tohoku University.

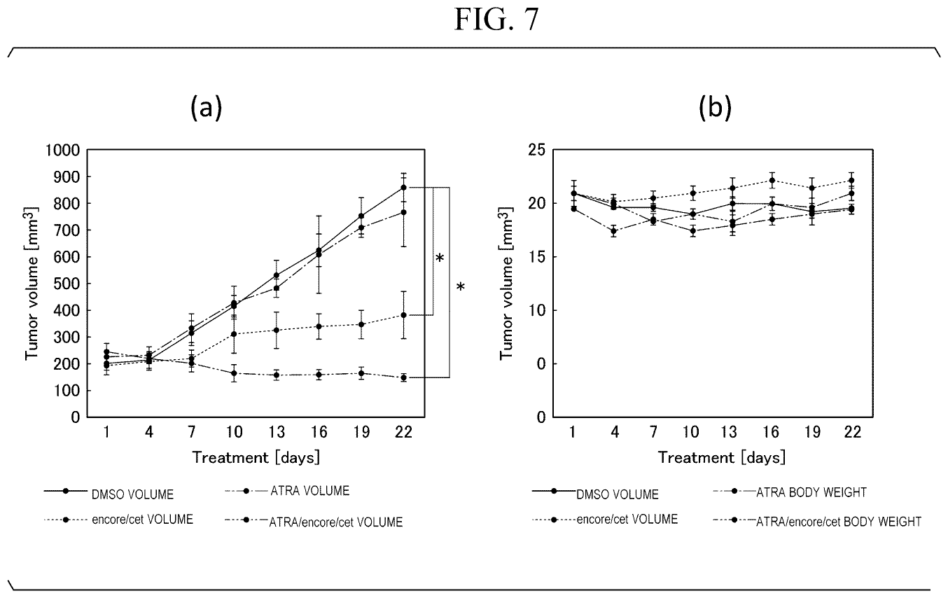

The results are shown in FIG. 7. (a) of FIG. 7 shows an average tumor volume, and (b) of FIG. 7 shows an average body weight. The significant difference in (a) of FIG. 7 was p<0.05 in One-way ANOVA and Tukey-Kramer test.

As shown in (a) of FIG. 7, in the groups in which only DMSO (control solvent) and ATRA were respectively administered, the volume (tumor size) increased with day, and the proliferation of tumor cells occurred. In the Encorafenib/Cetuximab administration group (enco/cet volume), an increase in tumor size is inhibited as compared with the above-described group, and an effect of inhibiting proliferation of Encorafenib/Cetuximab occurs as commonly known.

In contrast, a decrease in tumor size is observed in the ATRA/Encorafenib/Cetuximab administration group (ATRA/encore/cet volume). This shows that the administration of ATRA causes a synergistic effect with Encorafenib/Cetuximab, and the proliferation inhibition effect is remarkable to the extent that the tumor size decreases. As shown in (b) of FIG. 7, there is no major difference in body weight among the administration groups, and ATRA does not cause any significant side effects.

From these results, it was revealed that the components according to the embodiment in which ATRA was used in combination with encorafenib and cetuximab had a remarkable effect of reducing the tumor size even in the case of In Vivo, and were effective as a therapeutic agent for cancer.

### Test Example 6: Verification 2 of Cell Proliferation Inhibition Effect of Each Retinoid Compound

The effect of enhancing the cell proliferation inhibition effect, that is, the cell proliferation inhibition action of the BRAF inhibitor and the MEK inhibitor, was verified in more detail with a concentration smaller than that of Test Example 3 by using compounds for acting on a retinoid receptor other than ATRA. As the compounds for acting on a retinoid receptor, retinol, tamibarotene, and bexarotene among the retinoids were used. As the cell lines, the colorectal cancer cell lines including RKO, HT29, CO115, WiDR, COLO205, and LIM2405 described above were used. As the BRAF inhibitor and the MEK inhibitor to be used in combination, a component (EB) obtained by mixing encorafenib and binimetinib in equal amounts was used as in Test Example 2.

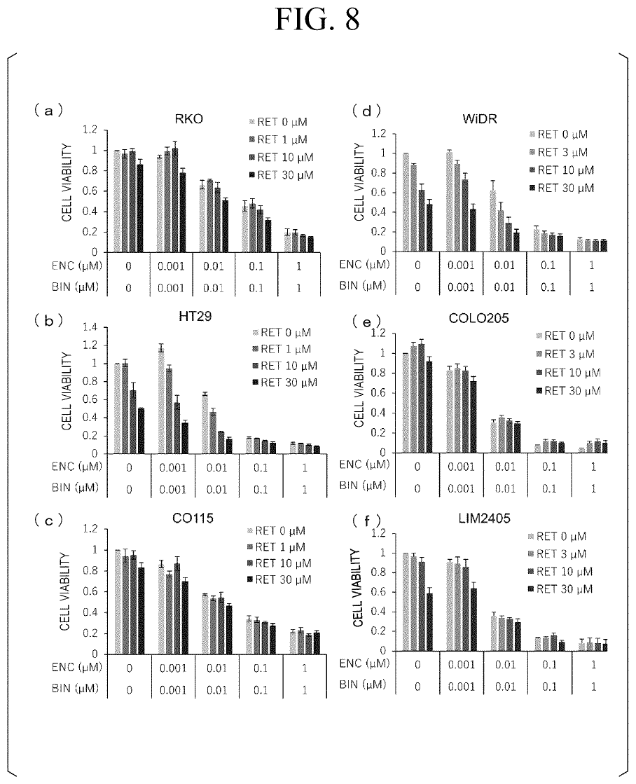

FIG. 8 shows results obtained by using retinol. (a) of FIG. 8 shows results obtained by using the RKO cell line, (b) of FIG. 8 shows results obtained by using the HT29 cell line, (c) of FIG. 8 shows results obtained by using the CO115 cell line, (d) of FIG. 8 shows results obtained by using the WiDR cell line, (e) of FIG. 8 shows results obtained by using the COLO205 cell line, and (f) of FIG. 8 shows results obtained by using the LIM2405 cell line.

As shown in these drawings, the cell viability decreased depending on the EB concentration in any cell line of RKO, HT29, CO115, WiDR, COLO205, and LIM2405. In addition, the cell viability was greatly decreased, the decrease in the cell viability was dependent on the concentration of retinol, and the effect of enhancing the cell proliferation inhibition action of EB was exhibited in the case of further adding retinol to EB.

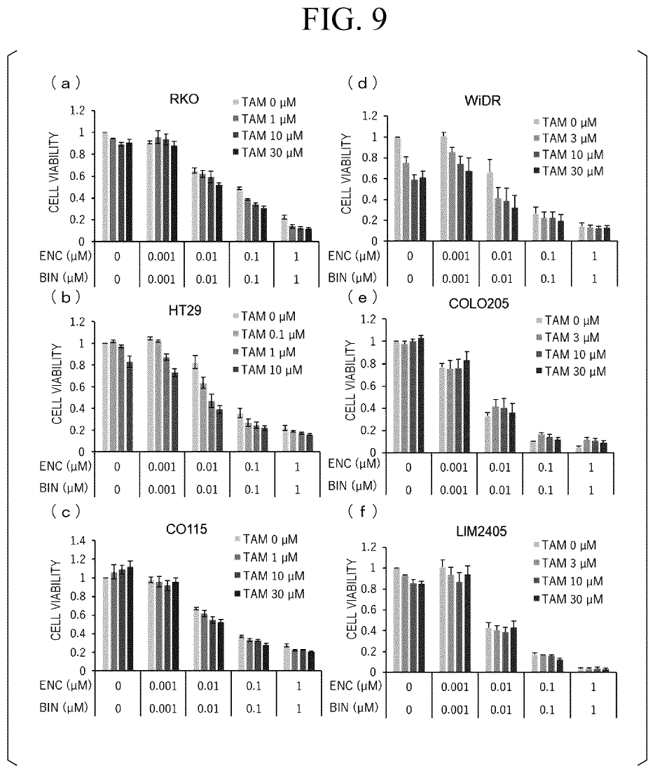

FIG. 9 shows results obtained by using tamibarotene. (a) of FIG. 9 shows results obtained by using the RKO cell line, (b) of FIG. 9 shows results obtained by using the HT29 cell line, (c) of FIG. 9 shows results obtained by using the CO115 cell line, (d) of FIG. 9 shows results obtained by using the WiDR cell line, (e) of FIG. 9 shows results obtained by using the COLO205 cell line, and (f) of FIG. 9 shows results obtained by using the LIM2405 cell line.

As shown in these drawings, the cell viability decreased depending on the EB concentration in any cell line of RKO, HT29, CO115, WiDR, COLO205, and LIM2405. In addition, the cell viability was greatly decreased, the decrease in the cell viability was dependent on the concentration of tamibarotene, and the effect of enhancing the cell proliferation inhibition action of EB was exhibited in the case of further adding tamibarotene to EB.

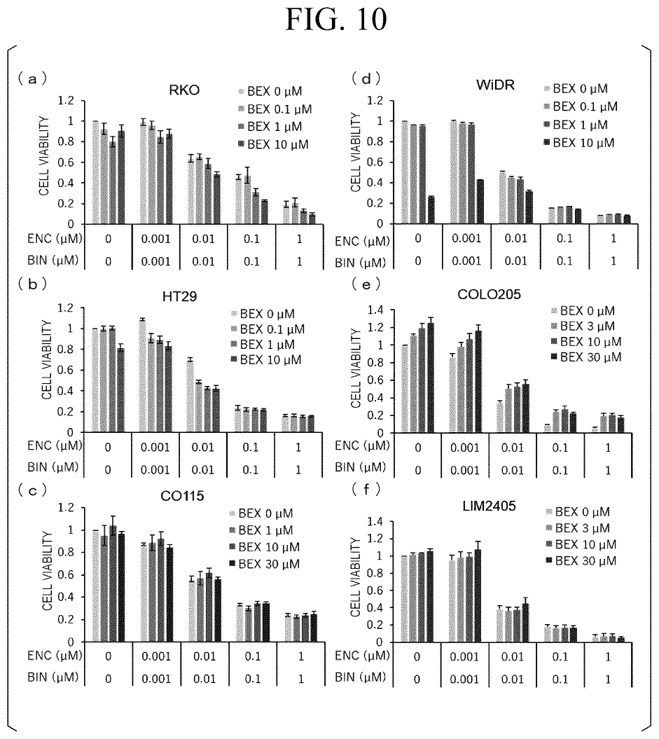

FIG. 10 shows results obtained by using bexarotene. (a) of FIG. 10 shows results obtained by using the RKO cell line, (b) of FIG. 10 shows results obtained by using the HT29 cell line, (c) of FIG. 10 shows results obtained by using the CO115 cell line, (d) of FIG. 10 shows results obtained by using the WiDR cell line, (e) of FIG. 10 shows results obtained by using the COLO205 cell line, and (f) of FIG. 10 shows results obtained by using the LIM2405 cell line.

As shown in these drawings, the cell viability decreased depending on the EB concentration in any cell line of RKO, HT29, CO115, WiDR, COLO205, and LIM2405. In addition, the cell viability was greatly decreased, the decrease in the cell viability was dependent on the concentration of bexarotene, and the effect of enhancing the cell proliferation inhibition action of EB was exhibited in the case of further adding bexarotene to EB.

From the above results, it was shown that retinol, tamibarotene, and bexarotene synergistically enhanced cell proliferation inhibition caused by EB in a plurality of BRAF-mutated colorectal cancer cell lines.

### Test Example 7: Verification of Influence of Combined Use of Compound Acting on Retinoid Receptor, BRAF Inhibitor, and MEK Inhibitor on Apoptosis

The influence of combined use of a compound acting on a retinoid receptor, a BRAF inhibitor, and an MEK inhibitor on induction of apoptosis in BRAF-mutated colorectal cancer cells was verified.

The RKO cell line and the HT29 cell line were respectively seeded on 6-well plates with the number of cells of 7.5×10⁴ cells/well and 8.0×10⁴ cells/well. After culturing for 24 hours, DMSO, TRE, ENC+BIN, or TRE+ENC+BIN were separately administered to each cell line, i.e., were separately administered to the RKO cell line to be DMSO (0.1 v/v %), TRE (10 μM), ENC (100 nM), and BIN (100 nM), and were separately administered to the HT29 cell line to be DMSO (0.1 v/v %), TRE (10 μM), ENC (10 nM), and BIN (10 nM), followed by culturing at 37° C. for 72 hours. Thereafter, the cells were collected, washed with PBS, and stained with Annexin V and PI using Annexin V-FITC Apoptosis Detection Kit (Nakalai Tesque). Apoptotic cells were detected using CytoFLEX LX. A proportion of cells positive for Annexin V was calculated as a proportion of apoptotic cells. The above was performed three times as an independent experiment. Student's test was used for a significant difference test.

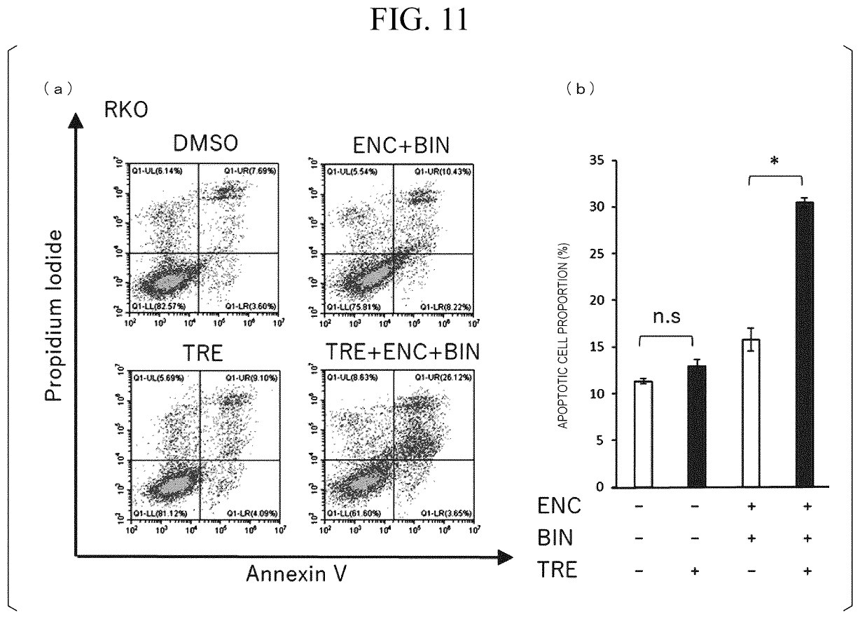

FIG. 11 shows results of an Annexin V-PI assay performed on the RKO cell line. (a) of FIG. 11 shows dot plots of propidium iodide (PI) for Annexin V in the cases of DMSO, TRE, ENC+BIN, and TRE+ENC+BIN. (b) of FIG. 11 shows an apoptotic cell proportion (%) in the cases of DMSO, TRE, ENC+BIN, and TRE+ENC+BIN.

In the RKO cell line, no significant change was observed in the apoptotic cell proportion in the case of DMSO and TRE. On the other hand, compared with ENC+BIN, the apoptotic cell proportion was significantly increased in the case of TRE+ENC+BIN.

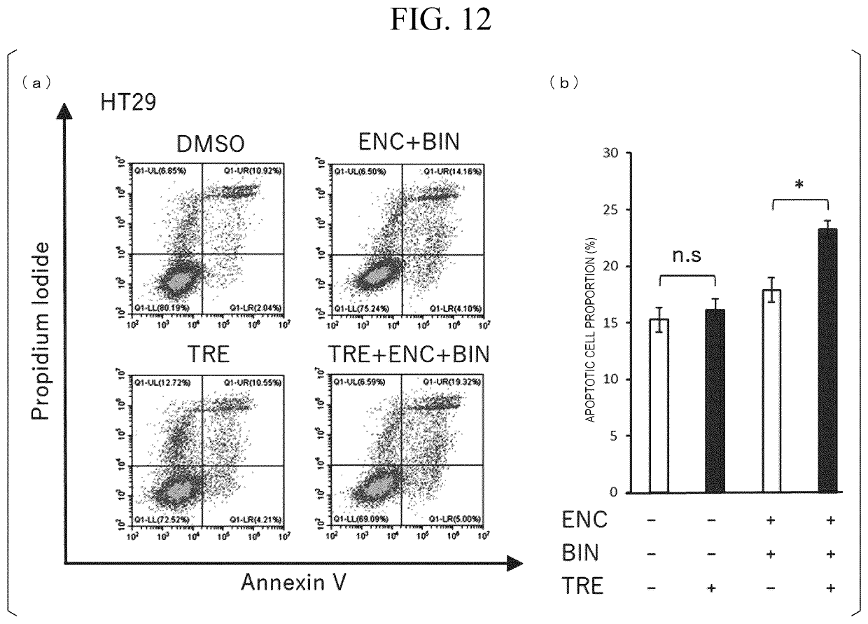

FIG. 12 shows results of an Annexin V-PI assay performed on the HT29 cell line. (a) of FIG. 12 shows dot plots of PI for Annexin V in the cases of DMSO, TRE, ENC+BIN, and TRE+ENC+BIN. (b) of FIG. 12 shows an apoptotic cell proportion (%) in the cases of DMSO, TRE, ENC+BIN, and TRE+ENC+BIN.

In the HT29 cell line, no significant change was observed in the apoptotic cell proportion in the case of DMSO and TRE. On the other hand, compared with ENC+BIN, the apoptotic cell proportion was significantly increased in the case of TRE+ENC+BIN.

From the above results, it was shown that the use of TRE in combination with ENC+BIN enhances the apoptosis inducing ability.

### Test Example 8: Analysis of Gene Expression Changing Depending on Single Use of Compound Acting on Retinoid Receptor and Combined Use of Compound Acting on Retinoid Receptor, BRAF Inhibitor, and MEK Inhibitor

In order to clarify the difference in gene expression changing depending on single use of a compound acting on a retinoid receptor alone and combined use of a compound acting on a retinoid receptor, a BRAF inhibitor, and an MEK inhibitor, comprehensive gene expression analysis was performed using an mRNA microarray.

The RKO cell line and the HT29 cell line were respectively seeded on 6-well plates with the number of cells of 5.0×10⁴ cells/well and 1.2×10⁵ cells/well. After culturing for 24 hours, DMSO, TRE, ENC+BIN, or TRE+ENC+BIN were separately administered to each cell line. To both cell lines, DMSO is administered to be 0.1 v/v %, TRE is administered to be 1 μM, ENC is administered to be 10 nM, and BIN is administered to be 10 nM, separately. The cells were collected after 24 hours after the drug administration, and total RNA was extracted using RNeasy Mini Kit (QIAGEN Inc.). An amount of the total RNA was measured using a NanoDrop ND-1000 spectrophotometer (Thermo Fisher Scientific Inc.). Total RNA of 100 ng extracted using Low Input Quick Amp Labeling Kit and one-color (Agilent Technologies) was amplified and labeled with Cyanine 3. The amount and quality of CRNA were measured using Agilent Bioanalyzer (Agilent Technologies) and NanoDrop One ND-ONE-W (Thermo Fisher Scientific Inc.). The labeled RNA was hybridized to Sure Print G3 Human GE Microarray 8*60K Ver. 3.0 (Agilent Technologies) by rotation at 65° C. for 17 hours. After washing, the microarray was scanned with Agilent DNA microarray scanner G2505C (Agilent Technologies). The fluorescence intensity obtained by scanning was quantified using Agilent Feature Extraction software version 10.7. Expression analysis was performed using Spring ver. 14.5 (Agilent Technologies). Normalization was performed by using a 75th percentile shift. The Sure Print G3 Human GE microarray 8*60K Ver 3.0 has a total of 50,599 probes. In this test, in order to specify significant differences in gene expression changes, a fold change of 2 times or more was used as a criterion for a significant difference.

Next, the Pathway analysis for a significant change in gene expression was performed using bioinformatics software (http://david.abcc.ncifcrf.gov/) called DAVID (the Database for Annotation, Visualization and Integration Discovery). Kyoto Encyclopedia of Genes and Genomes (KEGG) pathways that had p≤0.05 and contained 10 or more genes were extracted using the modified Fisher's exact test. Further, in order to examine whether there is a change in a molecular signaling pathway in a tumor, molecular signaling pathways belonging to KEGG Category 3 Environmental Information Processing and Category 4 Cellular Processes were extracted.

The number of genes of the RKO cell line, which were significantly changed depending on TRE and TRE+ENC+BIN and could be analyzed, were 790 and 1, 222, respectively. When the Pathway analysis was performed using these variable genes, three signaling pathways were concentrated in the case of TRE, and fourteen signaling pathways were concentrated in the case of TRE+ENC+BIN (see Tables 1 and 3 below). Further, in order to focus on the molecular signaling pathway in the tumor, the signaling pathways belonging to Environmental Information Processing and Cellular Processes in the KEGG category were limited, and three signaling pathways in the case of TRE and six signaling pathways in the case of TRE+ENC+BIN were identified.

TABLE 1

| RKO Cells | | | |
|---|---|---|---|
| KEGG pathway name | Number of genes | p value | Gene name |
| Cytokine-cytokine | 20 | 7.68909E−05 | GDF10, IL33, EDA, IL31, |
| receptor interaction | | | CD70, GDF15, IL26, CSH1 |
| | | | CCL4L2, EBI3, OSM, INHBA, |
| | | | GH1, IL1RL2, CXCL12, THP0, |
| | | | CLCF1, LTB, INHA, CCL26 |
| Calcium signaling | 12 | 0.028945839 | FGF17, PDE1C, GDNF, TBXA2R, |
| pathway | | | NTRK3, HGF, CAMK4, TNNC1 |
| | | | ORAI3, PDGFB, FGF1, GRIN2C |
| NF-kappa B | 7 | 0.038093938 | PIAS4, CXCL12, EDA, CCL4L2, |
| signaling pathway | | | LY96, TAB2, LTB |

TABLE 2

| HT29 Cells | | | |
|---|---|---|---|
| KEGG pathway name | Number of genes | p value | Gene name |
| Cytokine-cytokine | 25 | 0.004973311 | CCL14, CCL13, CD40, CSF2, |
| receptor interaction | | | CSH1, EBI3, MPL, CSF2RB, |
| | | | IL2RG, CXCR3, LEPR, CCL1 |
| | | | CCL15, CCL25, IL15RA, CCL20, |
| | | | GDF15, GDF9, CD4, CXCL11, |
| | | | CXCL12, IL1B, ACKR4, CD27, |
| | | | IL17C |
| Viral protein interaction | 11 | 0.017026132 | CCL25, CCL14, CCL13, CXCL11, |
| with cytokine and | | | CXCL12, CCL20, CXCR3, ACKR4, |
| cytokine receptor | | | CCL1, IL2RG, CCL15 |
| Neuroactive ligand- | 26 | 0.02893139 | CHRNA1, ADCYAP1R1, CSH1 |
| receptor interaction | | | LPAR1, NR3C1, GHRHR, GRM2, |
| | | | GLRA3, EDNRB, P2RY4, NPVF, |
| | | | LEPR, DRD5, PTGIR, EDN1 |
| | | | EDN2, TAAR8, GRID1, FSHB, |
| | | | NPY1R, SSTR1, GIP, GAL, |
| | | | LYPD6B, FSHR, F2RL2 |

TABLE 3

| RKO Cells | | | |
|---|---|---|---|
| KEGG pathway name | Number of genes | p value | Gene name |
| Cytokine-cytokine | 27 | 0.000492 | IL26, CCL4L2, CSH1, EB13 |
| receptor interaction | | | CSF2RA, IL18RAP, THP0, IL12RB2, |
| | | | GDF10, IL11, IL33, IFNB1, CD70, |
| | | | IL15, GDF15, TNFSF15, OSM, |
| | | | INHBA, TNFRSF1A, GH1, CXCL12, |
| | | | CLCF1, IL2RB, INHA, PF4, IL17A, |
| | | | CCL26 |
| JAK-STAT | 18 | 0.000694 | PIAS4, IL11, IFNB1, IL15, CSH1, |
| signaling pathway | | | PDGFB, OSM, PDGFA, PIK3CD, |
| | | | CSF2RA, GFAP, GH1, CCND3, |
| | | | SOCS1, THP0, IL2RB, PIM1, |
| | | | IL12RB2 |
| PI3K-Akt | 28 | 0.003267 | PHLPP1, TNXB, ITGB5, IRS1, |
| signaling pathway | | | CSH1, PDGFB, PDGFA, PIK3CD, |
| | | | LAMC2, AREG, CCND3, FGF9, |
| | | | CREB3L3, CD19, MYB, SPP1, |
| | | | ITGB7, LAMB3, IFNB1, HGF, OSM, |
| | | | PGF, FGF17, GH1, PPP2R2B, |
| | | | IL2RB, MTCP1, EPHA2 |
| Focal adhesion | 18 | 0.006862 | TNXB, LAMB3, ITGB5, CAV3, HGF, |
| | | | PDGFB, PDGFA, PIK3CD, LAMC2 |
| | | | PGF, CCND3, MYL7, SPP1, MYL10, |
| | | | FLNB, ITGB7, PPP1R12B, TLN1 |
| Calcium signaling | 19 | 0.017574 | PDE1C, HGF, NTRK3, TNNC1 |
| pathway | | | TNNC2, PDGFB, PDGFA, ATP2A1, |
| | | | GRIN2C, FGF17, P2RX5, GDNF, |
| | | | FGF9, TBXA2R, CAMK4, ORAI3, |
| | | | PLCE1, PLCG1, PLCB1 |
| TNF signaling | 11 | 0.024393 | RPS6KA5, CREB3L3, IFNB1, IL15, |
| pathway | | | IRF1, MMP3, BCL3, PIK3CD, TAB2, |
| | | | MAPK13, TNFRSF1A |

TABLE 4

| KEGG pathway name | Number of genes | p value | Gene name |
|---|---|---|---|
| Cytokine-cytokine | 45 | 1.45E−07 | CCL14, CSF1R, IFNA4, BMP10, CNTF, |
| receptor interaction | | | CXCL9, CD40, TNFRSF6B, CSF2, |
| | | | CXCL8, CCL3L3, IL24, TNFRSF11B, |
| | | | TNFRSF13C, CXCL3, IL2RG, IFNA8, |
| | | | CXCL16, THP0, CXCR3, IL21R, |
| | | | TNFSF11, CCL19, CCL15, TGFB2, |
| | | | IL4R, TGFB3, CCL20, IL37, GDF15, |
| | | | TNFSF15, IL34, IL1R2, OSM, BMP7, |
| | | | PRLR, IL22RA1, GDF9, CD4, CXCL11, |
| | | | IL7, IL1B, ACKR4, LTB, CRLF2 |
| Viral protein interaction | 17 | 0.000668 | CCL14, CSF1R, CXCL9, CXCL8, IL37, |
| with cytokine and | | | CCL20, IL34, CCL3L3, IL24, CXCL3, |
| cytokine receptor | | | IL2RG, IL22RA1, CXCL11, CXCR3, |
| | | | ACKR4, CCL19, CCL15 |
| Neuroactive ligand- | 39 | 0.002167 | GABRB3, NPSR1, VIPR1, PRSS1, |
| receptor interaction | | | LPAR1, GRIK2, LYPD6, C3, GRM2, |
| | | | GLRA3, TBXA2R, P2RY2, NPVF, NPY, |
| | | | PRSS3, DRD2, DRD3, DRD5, PTGIR, |
| | | | EDN2, GRID1, GCGR, HTR1D, GABRA3, |
| | | | NPY1R, PTH2R, CCK, SSTR1, PRLR, |
| | | | ADRA2A, AGT, APLN, GAL, ADORA2B, |
| | | | LYPD6B, FSHR, MC5R, APLNR, NMUR2 |
| CAMP signaling pathway | 25 | 0.009005 | SUCNR1, CALML5, PIK3CD, GLI1, |
| | | | NPY, FFAR2, DRD2, PRKACB, DRD5, |
| | | | ABCC4, JUN, EDN2, PDE4D, PTCH1, |
| | | | HTR1D, NPY1R, ATP2B4, SSTR1, FOS, |
| | | | MAPK10, TIAM1, FXYD2, FSHR, |
| | | | PPP1R1B, CREB5 |
| JAK-STAT signaling | 19 | 0.017723 | PDGFRB, IFNA4, CDKN1A, CNTF, |
| pathway | | | IL4R, CSF2, IL24, OSM, PIK3CD, |
| | | | IL2RG, PRLR, IFNA8, IL22RA2, |
| | | | SOCS2, IL22RA1, THP0, IL7, IL21R, |
| | | | CRLF2 |
| Ras signaling pathway | 25 | 0.01908 | CSF1R, CALML5, PIK3CD, TGFA, |
| | | | RASAL3, ETS1, RASGRP3, FGF5, |
| | | | FGF7, PAK6, PRKAGB, PDGFRB, |
| | | | PLA2G2D, RASA4B, KSR1, FLT3LG, |
| | | | MAPK10, TIAM1, MRAS, RASA4, |
| | | | FGF19, FGF18, GNB3, GNB5, FGFR3 |
| PI3K-Akt signaling | 34 | 0.022371 | CSF1R, IFNA4, CDKN1A, TNXB, |
| pathway | | | ITGA2B, TNC, LPAR1, PIK3CD, TGFA, |
| | | | IL2RG, IFNA8, PIK3CG, FGF5, COMP, |
| | | | TCL1B, FGF7, ITGB7, PDGFRB, IL4R, |
| | | | ITGA1, OSM, FLT3LG, PRLR, LPAR5, |
| | | | IL7, COL6A2, FGF19, FGF18, GNB3, |
| | | | COL9A3, GNB5, SGK1, FGFR3, CREB5 |
| ABC transporters | 8 | 0.024888 | ABCC4, ABCA2, ABCA10, ABCB1, |
| | | | ABCC2, TAP2, TAP1, DEFB1 |
| Wnt signaling pathway | 19 | 0.027444 | TLE4, JUN, FZD2, PRICKLE1, AXIN2, |
| | | | TLE6, PRICKLE4, NKD1, MAPK10, |
| | | | WNT11, DAAM2, BAMBI, FRZB, TBL1Y, |
| | | | TBL1X, LGR5, PLCB2, PRKACB, WNT4 |

It was shown that a gene changing depending on TRE+ENC+BIN was contained in a gene group belonging to a signaling pathway associated with cell proliferation, such as a JAK-STAT signaling pathway and a PI3K-Akt signaling pathway, which was not recognized in the case of TRE.

The genes of the HT29 cell line, which were significantly changed depending on TRE and TRE+ENC+BIN and could be analyzed, were 1,274 genes and 1, 619 genes, respectively. When the pathway analysis was performed using these variable genes, 12 signaling pathways were enriched in the case of TRE, and 31 signaling pathways were enriched in the case of TRE+ENC+BIN (see Tables 2 and 4). The analysis was performed in the same manner as the analysis of the RKO cell line, and finally, three signaling pathways were identified in the case of TRE, and nine signaling pathways were identified in the case of TRE+ENC+BIN. It was shown that a gene changing depending on TRE+ENC+BIN was contained in a gene group belonging to a molecular signaling pathway associated with cell proliferation, such as a JAK-STAT signaling pathway, a Ras signaling pathway, and a PI3K-Akt signaling pathway, which was not recognized in the case of TRE.

### Test Example 9: Analysis of Protein Expression Changing Depending on Combined Use of Compound Acting on Retinoid Receptor, BRAF Inhibitor, and MEK Inhibitor

In order to clarify the influence of the combined use of a compound acting retinoid receptor, a BRAF inhibitor, and an MEK inhibitor on the expression of a specific protein or a phosphorylated protein, comprehensive gene expression analysis was performed using an antibody microarray.

The RKO cell line was seeded on a 6-well plate with the number of cells of 5.0×10⁴ cells/well. After culturing for 24 hours, DMSO, TRE, ENC+BIN, or TRE+ENC+BIN were separately administered. DMSO was administered to be 0.1 v/v %, TRE was administered to be 10 μM, ENC was administered to be 10 nM, and BIN was administered to be 10 nM separately. The cells were collected after 48 hours after the drug administration. The cells were separated by centrifugation to prepare cell pellets, and the cell pellets were frozen at −80° C. The experimental steps after protein extraction were performed by the contract analysis of Kinex™ antibody microarray contract service (Cosmo Bio Co., Ltd.). The antibody array KAM-2000 (Kinexus Bioinformatics) used in this test targets 875 phosphorylation site-specific antibodies and 451 pan-specific antibodies. In this test, in order to specify a significant difference in changes of expression of the protein or the phosphorylated protein, a change of % Change From Control value (% CFC)≥45 and % CFC≤−45 recommended by Kinexus Bioinformatics was used as a criterion for a significant difference.

Next, the Pathway analysis for the changes in expression of the protein or the phosphorylated protein was performed using bioinformatics software (http://david.abcc.ncifcrf.gov/) called DAVID (the Database for Annotation, Visualization and Integration Discovery). Kyoto Encyclopedia of Genes and Genomes (KEGG) pathways that had p≤0.05 and contained 10 or more proteins or phosphorylated proteins were extracted using the modified Fisher's exact test. Further, in order to examine whether there is a change in a molecular signaling pathway in a tumor, molecular signaling pathways belonging to Environmental Information Processing in KEGG Category 3 and Cellular Processes in Category 4 were extracted.

In order to extract proteins and phosphorylated proteins specifically varied in the case of TRE+ENC+BIN, proteins and phosphorylated proteins varied in the case of TRE, and ENC+BIN was excluded from proteins varied in TRE+ENC+BIN. As a result, the number of analyzable proteins was 106. When the Pathway analysis was performed using these proteins and phosphorylated proteins, 30 signaling pathways were enriched. In order to focus on the molecular signaling pathway in the tumor, the signaling pathways belonging to Environmental Information Processing and Cellular Processes in the KEGG category were limited, and nine signaling pathways were identified (see Table 5 below).

TABLE 5

| | Number of | | |
|---|---|---|---|
| KEGG pathway name | proteins | p value | uniprot ID |
| MAPK signaling | 22 | 1.32E−12 | P51452, Q12852, P35968, Q15303, |
| pathway | | | P23560, P04626, P31749, O95382, |
| | | | P04629, Q02750, P10721, P31751, |
| | | | P15336, P10398, Q00653, P22736, |
| | | | P15056, Q16690, Q04206, P01106, |
| | | | P08069, O95819 |
| PI3K-Akt | 23 | 5.75E−12 | P35968, Q15303, P23560, P31946, |
| signaling pathway | | | P10415, P04626, P31749, P04629, |
| | | | Q02750, P10721, P31751, P15336, |
| | | | P54646, P22736, Q04206, P23588, |
| | | | P24941, P01106, P08069, P23458, |
| | | | P23443, Q13131, Q04917 |
| Fox0 signaling | 12 | 9.36E−08 | P15056, Q13315, P31749, P24941, |
| pathway | | | P08069, Q02750, Q9H4B4, P31751, |
| | | | Q9NYY3, Q13131, P54646, P10398 |
| ErbB signaling | 10 | 2.04E−07 | P15056, Q15303, P00519, P04626, |
| pathway | | | P31749, P01106, Q02750, P31751, |
| | | | P23443, P10398 |
| NF-kappa B | 10 | 1.16E−06 | Q00653, P43403, Q04759, P10415, |
| signaling pathway | | | Q04206, Q13315, Q13546, P07948, |
| | | | P09874, Q06187 |
| Autophagy - animal | 11 | 1.79E−06 | Q14457, Q9Y4P1, Q04759, P10415, |
| | | | P31749, P08069, Q02750, P31751, |
| | | | P23443, Q13131, P54646 |
| mTOR signaling | 10 | 3.24E−05 | P15056, P31749, P23588, P08069 |
| pathway | | | Q02750, P31751, P23443, Q13131, |
| | | | P54646, Q14693 |
| Ras signaling | 11 | 0.000153 | P43403, P23560, P35968, P00519, |
| pathway | | | Q04206, P31749, P04629, P08069, |
| | | | Q02750, P10721, P31751 |
| Focal adhesion | 10 | 0.000229 | P15056, P35968, P10415, P04626, |
| | | | P31749, P06241, P08069, Q02750, |
| | | | P35222, P31751 |

It was shown that the proteins and the phosphorylated proteins specifically varied depending on the TRE+ENC+BIN belonged to signaling pathways associated with tumor proliferation, such as an MAPK signaling pathway, a PI3K-Akt signaling pathway, a FoxO signaling pathway, and an ErbB signaling pathway.

### Test Example 10: Change in Protein Expression Depending on Combined Use of Compound Acting on Retinoid Receptor, BRAF Inhibitor, and MEK Inhibitor

In order to clarify a molecular biological mechanism relating to the enhancement of the cell proliferation inhibition effect of the combined use of a compound acting on a retinoid receptor, a BRAF inhibitor, and an MEK inhibitor, a change in protein expression by the combined use of the compound acting on a retinoid receptor, the BRAF inhibitor, and the MEK inhibitor was checked.

The type and dilution ratio of the primary antibody used in the Western blot are shown in Table 6 below.

TABLE 6
species

| | Host animal | | | |
|---|---|---|---|---|
| Antibody | species | Seller | Product number | Dilution ratio |
| PARP | Rabbit | Cell Signaling | 9532 | 1:2000 |
| | | Technology | | |
| Phospho-p44/42 MAPK | Rabbit | Cell Signaling | 4094 | 1:1000 |
| (Erk1/2) (Thr202/Tyr204) | | Technology | | |
| p44/42 MAPK (Erk1/2) | Rabbit | Cell Signaling | 9102 | 1:1000 |
| | | Technology | | |
| Phospho-Akt (Ser473) | Rabbit | Cell Signaling | 4060 | 1:1000 |
| | | Technology | | |
| Mcl-1 | Rabbit | Cell Signaling | 5453 | 1:1000 |
| | | Technology | | |
| BAX | Rabbit | Cell Signaling | 5023 | 1:1000 |
| | | Technology | | |
| BAK | Rabbit | Cell Signaling | 3814 | 1:1000 |
| | | Technology | | |
| Bcl-2 | Rabbit | Cell Signaling | 2876 | 1:1000 |
| | | Technology | | |
| Bcl-xL | Rabbit | Cell Signaling | 2764 | 1:1000 |
| | | Technology | | |
| Phospho-Histone H2A. X | Rabbit | Cell Signaling | 2577 | 1:1000 |
| (Ser139) | | Technology | | |
| RARα | Mouse | Santa Cruz | sc-515796 | 1:500 |
| | | Animal Health | | |
| RXRα | Mouse | Santa Cruz | sc-515929 | 1:1000 |
| | | Animal Health | | |
| β-Actin | Mouse | Sigma-Aldrich | A5316 | 1:1000 |
| α-tubulin | Mouse | Sigma-Aldrich | T5168 | 1:1000 |
| GAPDH | Mouse | Sigma-Aldrich | G8795 | 1:1000 |
| GAPDH | Rabbit | Sigma-Aldrich | PLA0125 | 1:1000 |

To the collected cells were added Radioimmunoprecipitation assay buffer (composition: 50 mM Tris-HCL, pH 8.0, 150 mM Sodium Chloride, 0.5 w/v % Sodium Deoxycholate, 0.1 w/v % Sodium Dodecyl Sulfate, 1.0 w/v % NP-40 substitute) supplemented with Halt Protease Inhibitor Cocktail (Thermo Fisher Scientific Inc.), followed by dissolving the cells, and a protein was extracted. The extracted protein was quantified by a bicinchoninic acid method. A protein sample in an amount of 5 μg was separated by polyacrylamide electrophoresis, and the separated protein sample was transferred to Polyvinylidene difluoride membrane (PVDF membrane, Merck Millipore Ltd.), followed by blocking with Odyssey Blocking Buffer (Licor Inc.) for 1 hour at room temperature. Thereafter, the PVDF membrane after transfer was immersed in a primary antibody solution, followed by culturing at room temperature for 2 hours or at 4° C. overnight. After the primary antibody reaction, the PVDF membrane was washed with Tris Buffered Saline with Tween 20 (TBS-T) for 5 minutes at room temperature for three times and immersed in a secondary antibody, followed by incubating at room temperature for 1 hour. As the secondary antibody, Goat anti-Rabbit IgG (H+L) Highly Cross-Adsorbed Secondary Antibody Alexa Fluor680 (Thermo Fisher Scientific Inc.) and Goat anti-Mouse IgG (H+L) Highly Cross-Adsorbed Secondary Antibody Alexa Fluor680 (Thermo Fisher Scientific Inc.) were used at a dilution ratio of 1:1000. Thereafter, washing with TBS-T for 5 minutes was performed three times, and a measurement of signals was performed using an Odyssey Infrared Imaging System (Licor Inc.). The detected band was quantified using densitometry according to the analysis software ImageJ version 1.53a (The National Institute of Health, available at: http://imagej.nih.gov/ij/), and the correction was performed by internal control β-Actin, α-tubulin, or GAPDH. The above was performed three or more times as independent experiments. Student's test was used for a significant difference test.

Based on the results of the mRNA microarray and the antibody array, the expression levels of p-ERK, t-ERK, and p-MEK associated with the MAPK signaling pathway, and p-AKT associated with the PI3K-Akt signaling pathway were analyzed. In addition, from the results of the Annexin in V-PI assay described above, TRE enhances the apoptosis inducing ability generated by ENC+BIN. One of the mechanisms of apoptosis induction by TRE is regulation of the Bcl-2 family, and therefore, the expression levels of Bcl-2, Mcl-1, Bcl-xL, BAX, and BAK which are Bcl-2 family proteins in addition to PARP were analyzed. Further, TRE induces DNA damage and tumor proliferation inhibition when used alone or in combination with cytotoxic anticancer drugs in therapy. It has also been reported that DNA damage and Bcl-2 family influence each other. From the past reports, the expression level of p-H2AX, which reflects DNA damage considered to be associated with Bcl-2 family-mediated apoptosis caused by TRE, was also analyzed.

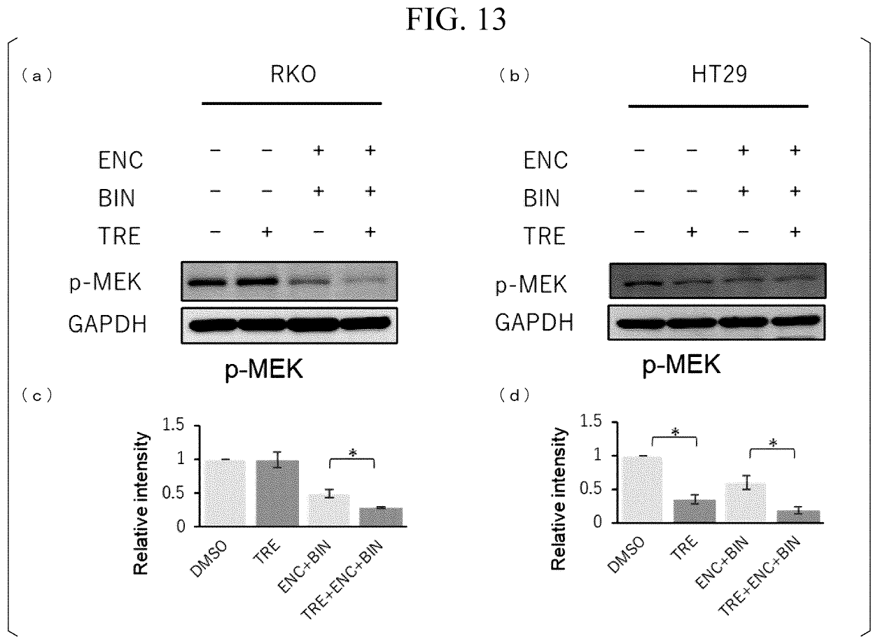

FIG. 13 shows results of analyzing an expression change of p-MEK related to the MAPK signaling pathway using a Western blot. (a) of FIG. 13 shows a Western blot band obtained by using the RKO cell line, (b) of FIG. 13 shows a Western blot band using the HT29 cell line, (c) of FIG. 13 is a graph in which a signal intensity of the band shown in (a) of FIG. 13 is quantified, and (d) of FIG. 13 is a graph in which a signal intensity of the band shown in (b) of FIG. 13 is quantified.

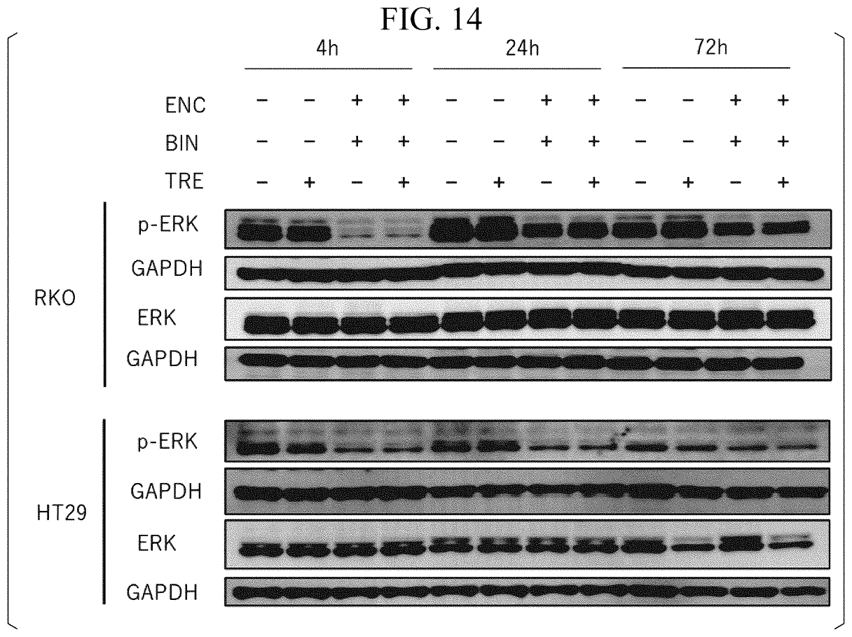

FIG. 14 shows results of analyzing an expression change of p-ERK and t-ERK related to the MAPK signaling pathway using a Western blot.

The combined use of TRE and ENC+BIN significantly reduced the expression of p-MEK in the RKO cell line and HT29 cell line as compared with ENC+BIN ((a) of FIG. 13 and (b) of FIG. 13). However, no significant difference was observed in the expression of p-ERK, which is downstream of p-ERK (FIG. 14). This result suggests that the mechanism for enhancing the cell proliferation inhibition by the combined use of TRE and ENC+BIN was not primarily mediated directly through the MAPK signaling pathway.

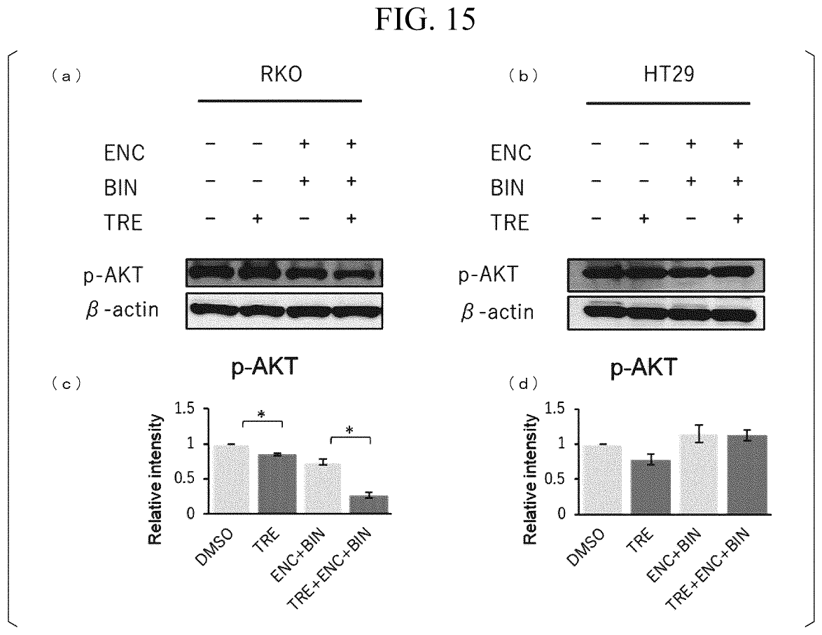

FIG. 15 shows results of analyzing an expression change of p-AKT related to the PI3K-Akt signaling pathway using a Western blot. (a) of FIG. 15 shows a Western blot band obtained by using the RKO cell line, (b) of FIG. 15 shows a Western blot band using the HT29 cell line, (c) of FIG. 15 is a graph in which a signal intensity of the band shown in (a) of FIG. 15 is quantified, and (d) of FIG. 15 is a graph in which a signal intensity of the band shown in (b) of FIG. 15 is quantified.

Although the combined use of TRE and ENC+BIN significantly reduced the expression of p-AKT in the RKO cell line as compared with ENC+BIN, the same tendency was not recognized in the HT29 cell line.

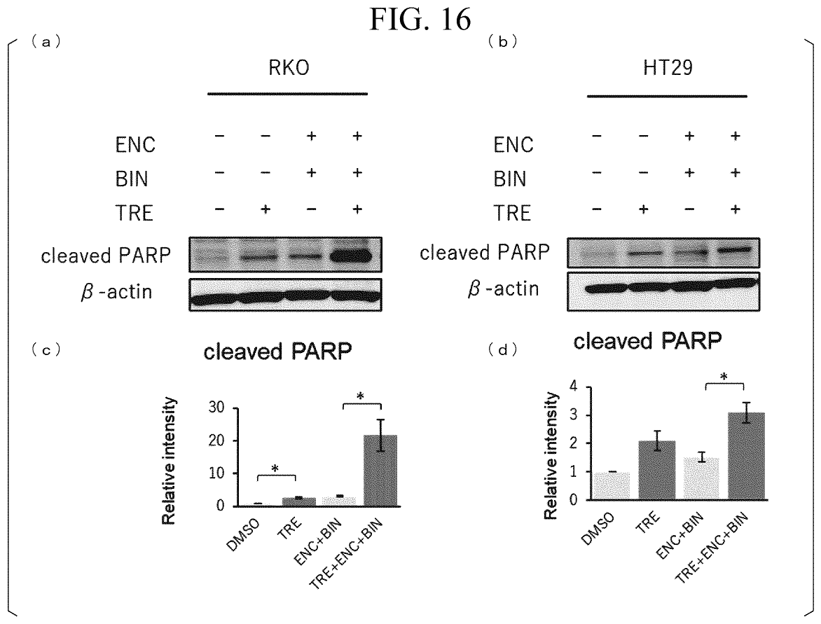

FIG. 16 shows results of analyzing an expression change of PARP related to a mechanism of apoptosis induction using a Western blot. (a) of FIG. 16 shows a Western blot band obtained by using the RKO cell line, (b) of FIG. 16 shows a Western blot band using the HT29 cell line, (c) of FIG. 16 is a graph in which a signal intensity of the band shown in (a) of FIG. 16 is quantified, and (d) of FIG. 16 is a graph in which a signal intensity of the band shown in (b) of FIG. 16 is quantified.

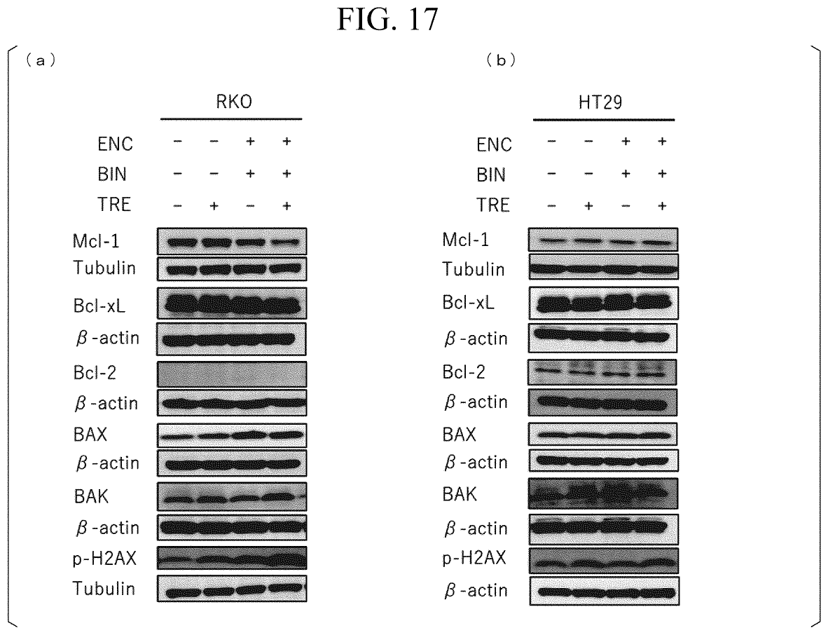

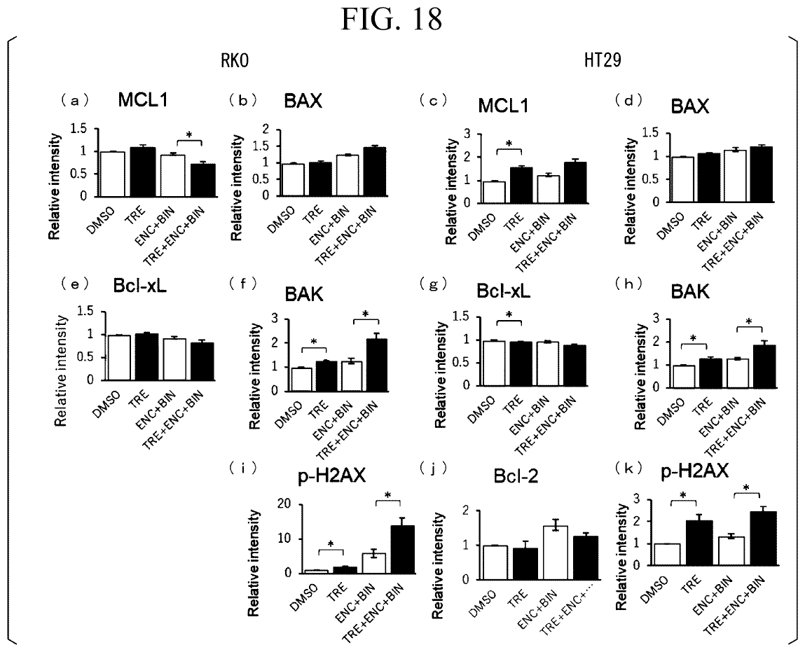

FIG. 17 shows results of analyzing an expression change of proteins related to the Bcl-2 family using a Western blot. (a) of FIG. 17 shows results obtained by using the RKO cell line, and (b) of FIG. 17 shows results obtained by using the HT29 cell line. FIG. 18 is a graph in which a signal intensity of a band of each protein shown in FIG. 17 is quantified. (a) of FIG. 18, (b) of FIG. 18, (e) of FIG. 18, (f) of FIG. 18, and (i) of FIG. 18 show results obtained by using the RKO cell line, and (c) of FIG. 18, (d) of FIG. 18, (g) of FIG. 18, (h) of FIG. 18, (j) of FIG. 18, and (k) of FIG. 18 show results obtained by using the HT29 cell line.

The combined use of TRE and ENC+BIN enhanced the expression of cleaved PARP, BAK and p-H2AX in the RKO cell line and HT29 cell line as compared with ENC+BIN. This result showed that the enhancement of the apoptosis inducing ability contributed to the molecular biological mechanism for enhancing the cell proliferation inhibition generated by the combined use of TRE and ENC+BIN, and supported results of an Annexin V-PI assay. It was also suggested that the combined use of TRE and ENC+BIN enhanced the expression of BAK, which is an effector of the Bcl-2 family, and p-H2AX, which is a marker of DNA damage, as compared with ENC+BIN, and the mechanism thereof was associated with the Bcl-2 family and DNA damage.

### Test Example 11: Verification of Relevance Between Effect of Enhancing Cell Proliferation Inhibition by Compound Acting on Retinoid Receptor and Protein Expression of Endogenous RARα and RXRα

Examples of receptors of TRE relevant to the cell proliferation inhibition effect of TRE include RARα and RXRα. It was assumed that examples of a mechanism of causing a compound acting on a retinoid receptor, such as TRE, to enhance the cell proliferation effect of ENC+BIN include a RARα or RXRα-mediated mechanism. First, in order to evaluate the relationship between the protein expression degree of endogenous RARα or RXRα and the effect of enhancing the cell proliferation inhibition of ENC+BIN by TRE, analysis was performed using a Western blot by the same method as in Test Example 10.

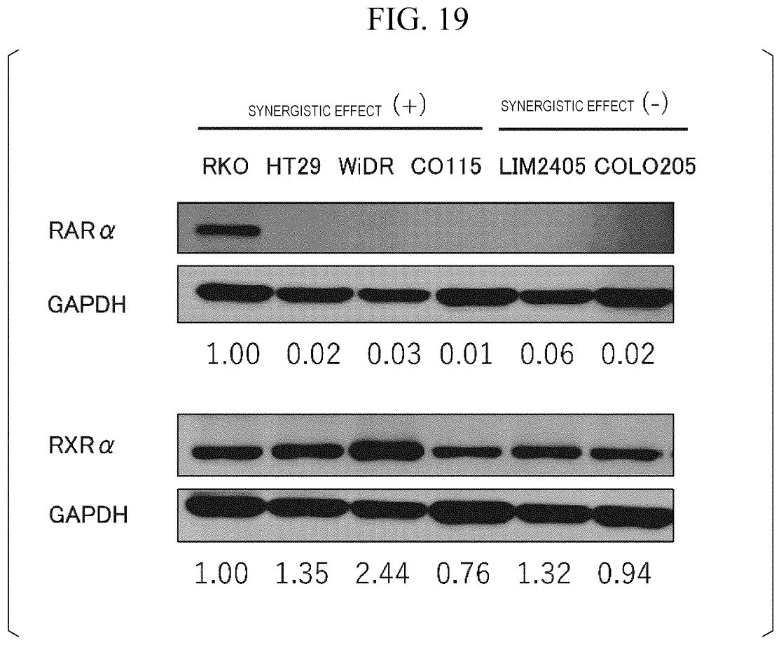

FIG. 19 shows results of analyzing the protein expression of endogenous RARα and RXRα in each BRAF-mutated colorectal cancer cell line by a Western blot.

The expression of endogenous RARα was recognized in the RKO cell line, but was hardly recognized in other cell lines. The expression of endogenous RXRα was increased in the order of the WIDR cell line, the HT29 cell line, the LIM2405 cell line, the RKO cell line, the COLO205 cell line, and the CO115 cell line.

From these results, no clear correlation was recognized between the effect of enhancing the cell proliferation inhibition of ENC+BIN by TRE and the protein expression levels of endogenous RARα and RXRα.

### Test Example 12: Verification of Cell Proliferation Inhibition Effect of Combined Use of Compound Acting on Retinoid Receptor, BRAF Inhibitor, and MEK Inhibitor Under Expression Regulation of RARα or RXRα

Next, in order to clarify whether the effect of enhancing the cell proliferation inhibition of a BRAF inhibitor and an MEK inhibitor by a compound acting on a retinoid receptor is influenced by the expression change of RARα or RXRα, an MTT assay was performed using the RKO cell line and the HT29 cell line in which the expression of RARα or RXRα was inhibited by siRNA.

Specifically, siGENOME Human RARA siRNA SMARTpool (catalog #M-003437-02-0005), siGENOME Human RXRA siRNA SMARTpool (catalog #M-003443-02-0005), and siGENOME Non-Targeting siRNA Pool (catalog #D-001206-13-05) among SiGENOME siRNA Reagents (Horizon Discovery Ltd.) were used for expression exhibition caused by siRNA. Using Lipofectamine™ 2000 Transfection Reagent (Thermo Fisher Scientific Inc.), transfection was performed under the condition that a final concentration of siRNA was 25 nM.

In consideration of the fact that both RARα and RXRα were expressed endogenously in RKO cells and only RXRα was expressed endogenously in other BRAF-mutated colorectal cancer cells, TRE, which acts on both RARα and RXRα, and BEX, a selective RXR agonist, among retinoids were used.

First, protein expression changes of RARα and RXRα of the RKO cell line generated by siRNA were analyzed using a Western blot by the same method as in Test Example 10. In Western blot analysis performed on RKO cells in which expression of RARα and RXRα was inhibited using siRNA, evaluation was performed using a relative value with DMSO in si-NC-introduced cells taken as 1.

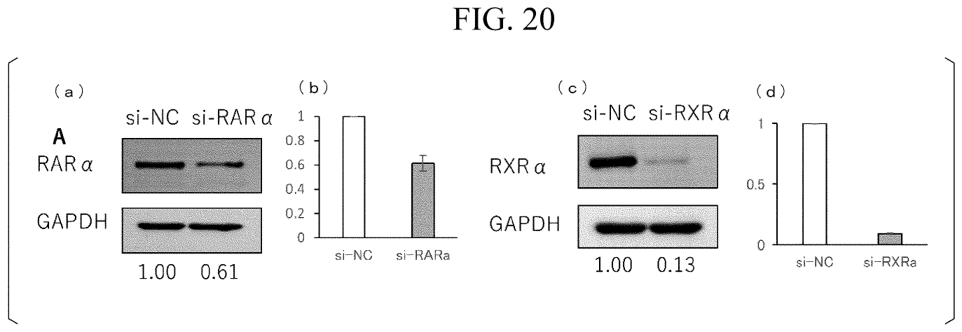

FIG. 20 shows results of analyzing the expression of RARα and RXRα in RKO cell lines under RARα or RXRα knockdown using a Western blot. (a) of FIG. 20 shows a band of a Western blot for an RKO cell line under RARα knockdown. (b) of FIG. 20 shows a graph in which the band shown in (a) of FIG. 20 is quantified. (c) of FIG. 20 shows a band of a Western blot for the RKO cell line under RXRα knockdown. (d) of FIG. 20 shows a graph in which the band shown in (c) of FIG. 20 is quantified.

It was confirmed that the expression of RARα decreased by 39±6% due to si-RARα and the expression of RXRα decreased by 87±2.3% due to si-RXRα as compared with si-NC as a control.

Then, DMSO (0.1 v/v %), TRE (10 μM), ENC (10 nM)+BIN (10 nM), TRE (10 μM)+ENC (10 nM)+BIN (10 nM), BEX (10 μM), and BEX (10 μM)+ENC (10 nM)+BIN (10 nM) were separately administered to each of the RKO cell lines into which siRNA or si-NC had been introduced, and the cell viability was evaluated after 72 hours after the administration.

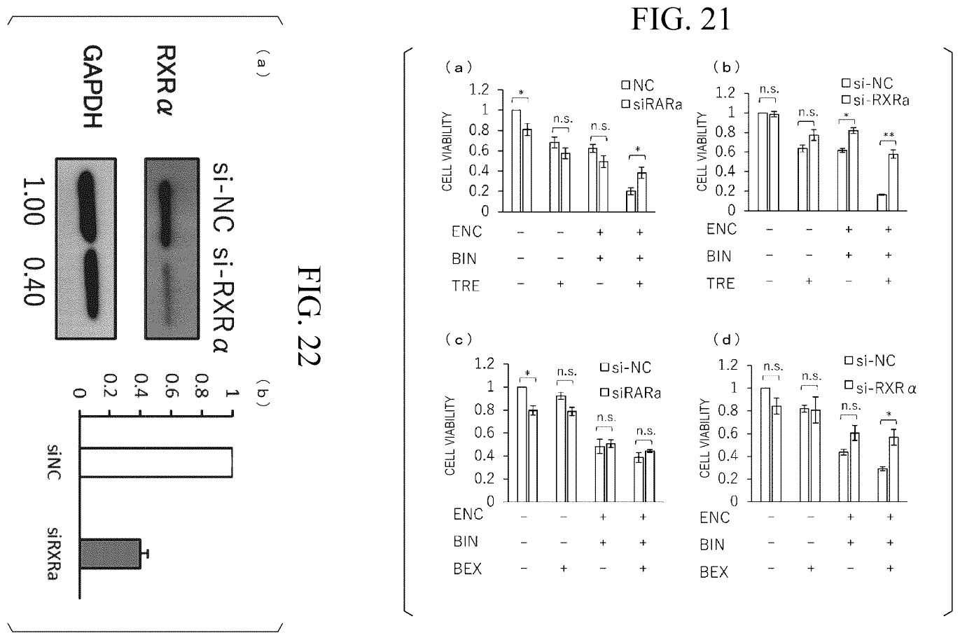

FIG. 21 shows results of examining proliferation inhibition effects of TRE+ENC+BIN or BEX+ENC+BIN in an RKO cell line under RARα or RXRα knockdown. (a) of FIG. 21 shows results of TRE+ENC+BIN in an RKO cell line under RARα knockdown, (b) of FIG. 21 shows results of TRE+ENC+BIN in an RKO cell line under RXRα knockdown, (c) of FIG. 21 shows results of BEX+ENC+BIN in an RKO cell line under RARα knockdown, and (d) of FIG. 21 shows results of BEX+ENC+BIN in an RKO cell line under RXRα knockdown.

The inhibition of RARα inhibited cell proliferation by 19±6% and 21±4% in the tests using TRE and BEX, respectively. On the other hand, when BEX, which is a selective RXR agonist, was used, the cell proliferation inhibition effect of BEX+ENC+BIN was reduced only by RXRα inhibition.

Next, the protein expression level of endogenous RARα in the HT29 cell line in the Western blot was low, and it was difficult to evaluate knockdown efficiency. Therefore, only the inhibition of RXRα in HT29 cells was evaluated using the same method as in the RKO cell line.

FIG. 22 shows results of analyzing expression of RXRα in a HT29 cell line under RXRα knockdown using a Western blot. (a) of FIG. 22 shows a band of a Western blot for the HT29 cell line under RXRα knockdown. (b) of FIG. 22 is a graph in which the band shown in (a) of FIG. 22 was quantified.

It was confirmed that the expression of RXRα decreased by 60±5% due to si-RXRα as compared with si-NC as a control.

Then, DMSO (0.1 v/v %), TRE (10 μM), ENC (10 nM)+BIN (10 nM), TRE (10 μM)+ENC (10 nM)+BIN (10 nM), BEX (10 μM), and BEX (10 μM)+ENC (10 nM)+BIN (10 nM) were separately administered to each of the HT29 cell line into which si-RXRα or si-NC had been introduced, and the cell viability was evaluated after 72 hours after the administration.

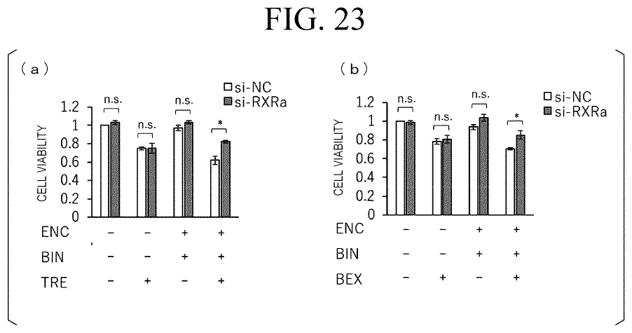

FIG. 23 shows results of examining proliferation inhibition effects of TRE+ENC+BIN or BEX+ENC+BIN in an RKO cell line under RXRα knockdown. (a) of FIG. 23 shows results of TRE+ENC+BIN under RXRα knockdown, and (b) of FIG. 23 shows results of BEX+ENC+BIN under RXRα knockdown.

The RXRα inhibition reduced cell proliferation inhibition effects of TRE+ENC+BIN or BEX+ENC+BIN, similar to the RKO cell line.

From these results, it was shown that the effect of enhancing the cell proliferation inhibition generated by ENC+BIN by retinoids was partially mediated through RARα or RXRα.

Next, in order to clarify whether the inhibition of RARα or RXRα influences the apoptosis caused by the combined use of TRE and ENC+BIN or ENC+BIN+CET, the expression level of cleaved PARP was analyzed using a Western blot by the same method as in Test Example 10.

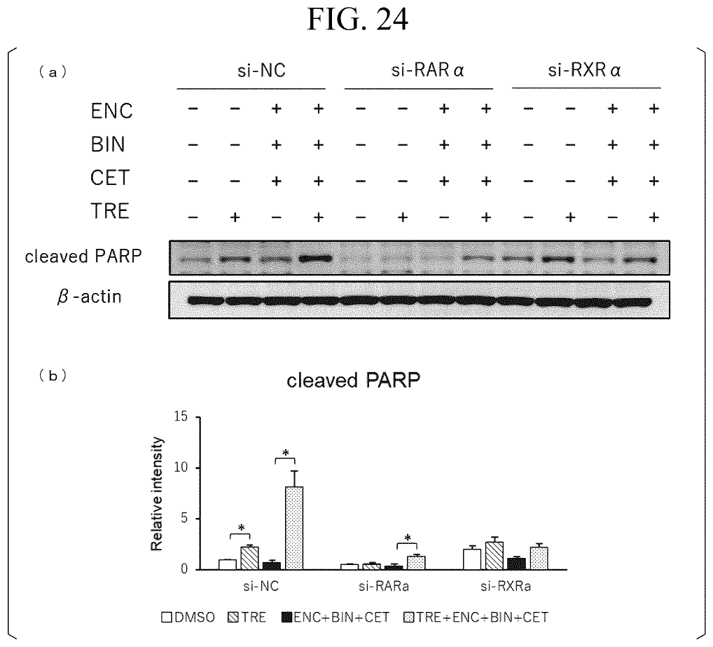

FIG. 24 shows results of analyzing expression of cleaved PARP caused by TRE+ENC+BIN+CET in an RKO cell line under RARα or RXRα knockdown. (a) of FIG. 24 shows a band of a Western blot in an RKO cell line under RARα or RXRα knockdown. (b) of FIG. 24 shows a graph in which the band shown in FIG. (a) of FIG. 24 is quantified.

In the cells into which si-NC was introduced, TRE enhanced the expression levels of cleaved PARP caused by ENC+BIN and ENC+BIN+CET to about 13 times and about 11 times, respectively. When RARα was inhibited, the effect of enhancing the expression levels of cleaved PARP caused by ENC+BIN and ENC+BIN+CET by TRE decreased to about 8 times and about 3 times, respectively. In addition, when RXRα is inhibited, the above expression levels were reduced to about 1.5 times and about 2 times. These results were considered to be consistent with the results of the MTT assay using siRNA.

From the above results, it was shown that, in the molecular biological mechanism in which the compound acting on a retinoid receptor, such as TRE, enhances the cell proliferation inhibition generated by ENC+BIN and ENC+BIN+CET, there is an enhancement in the apoptosis inducing ability partially mediated through RARα or RXRα.

### Test Example 13: Verification of Antitumor Effect of Combined Use of Compound Acting on Retinoid Receptor, BRAF Inhibitor, MEK Inhibitor, and Anti-EGFR Antibody in Subcutaneous Transplanted Tumor Mouse Model

The in vivo antitumor effect and toxicity generated by combined use of a compound acting on a retinoid receptor, a BRAF inhibitor, an MEK inhibitor, and an anti-EGFR antibody, or a BRAF inhibitor and an anti-EGFR antibody were evaluated by a mouse model subcutaneous transplanted with a HT29 cell line.

Female nude mice (BALB/c-nu) were purchased from The Jackson Laboratory Japan, and raised in a specific pathogen-free environment. The cultured HT29 cell line was collected with trypsin, and a culture medium and a Corning Matrigel basement membrane matrix (Corning) were mixed at a volume ratio of 1:1 so as to reach 1×10⁷ cells/mL, followed by suspension. Into a left abdominal region of each mouse was subcutaneously implanted 0.1 mL of cell suspension. When tumor volumes reached 150 mm³ to 200 mm³, mice were randomly divided into a vehicle group, a TRE single group, an ENC+CET group, a three-drug combination (TRE+ENC+CET) group including TRE and ENC+CET, an ENC+BIN+CET group, or a four-drug combination (TRE+ENC+BIN+CET) group including TRE and ENC+BIN+CET. TRE, ENC, and BIN were dissolved in Corn oil (FUJIFILM Wako Pure Chemical Industries, Ltd.), and further subjected to ultrasonic treatment using Bioruptor UCW-310 (Sonicbio. Co. Ltd.) to prepare a drug solution. ENC, BIN, and TRE were orally administrated by force once a day for 28 days at doses of 5 mg/kg, 1.75 mg/kg, and 10 mg/kg, respectively. CET was administered intraperitoneally twice a week at a dose of 20 mg/kg. Each solvent was used as the control. The tumor size and the body weight were measured every three days from the start of the therapy. The tumor volume was estimated by a calculation formula “tumor volume (mm³)=½×major axis (mm)×minor axis (mm)”. The animal experiment was approved by the Institutional Animal Care and Use Committee of the Tohoku University, and was performed according to the facility guideline of the Tohoku University.

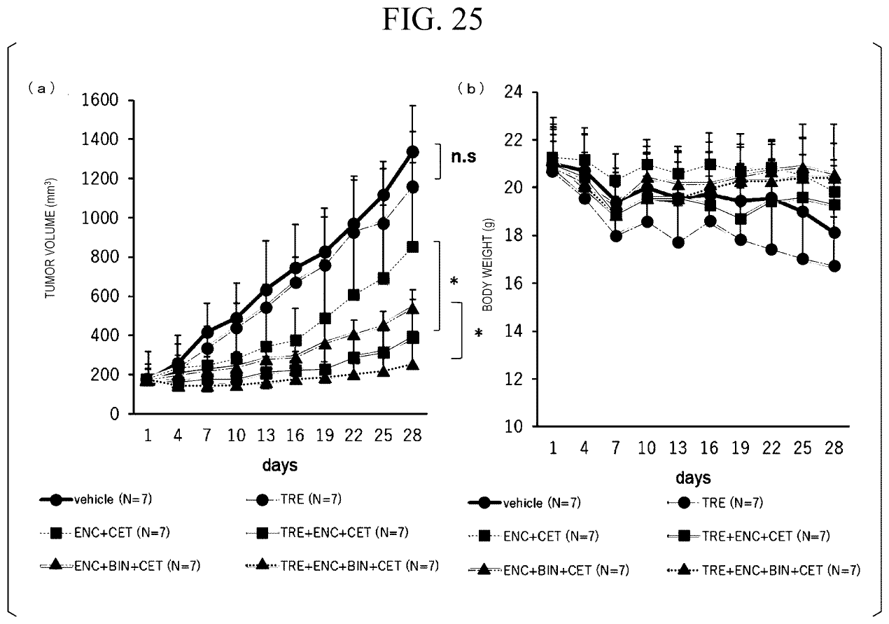

FIG. 25 shows results of examining an antitumor effect of the combined use of TRE and ENC+BIN+CET or ENC+CET in the subcutaneous transplanted tumor mouse model. (a) of FIG. 25 shows a temporal change in tumor volume (mm³), and (b) of FIG. 25 shows a temporal change in body weight (g).

The tumor volume of the vehicle group at a time point of 28 days from the start of the therapy was 1339 mm³±231 mm³, whereas the tumor volume of the TRE single group was 1156 mm³±283 mm³, and no significant reduction was recognized. On the other hand, the tumor volumes of the ENC+BIN+CET group and the ENC+CET group were 854 mm³±425 mm³ and 543 mm³±88 mm³, respectively, whereas the tumor volumes of the TRE+ENC+BIN+CET group and the TRE+ENC+CET group were 389 mm³±196 mm³ and 255 mm³±111 mm³, respectively, and significant reduction of 54% and 53% were recognized respectively.

This result shows that TRE enhances the in vivo antitumor effect of ENC+BIN+CET or ENC+CET.

In order to evaluate toxicity generated by TRE, the body weight of the mice in the therapy group was measured as shown in (b) of FIG. 25. The body weights of the vehicle group and the TRE single group, the body weights of the ENC+CET group and the TRE+ENC+CET group, and the body weights of the ENC+BIN+CET group and the TRE+ENC+BIN+CET group at the time point of 28 days after the therapy were respectively compared. As a result, there was no significant difference in mouse body weights depending on the presence or absence of TRE.

Subsequently, in order to evaluate the influence of the combined use of TRE and ENC+BIN+CET or ENC+CET on in vivo tumor proliferation, a Ki-67 labeling index was calculated using a resected tumor at the end of the therapy.

Specifically, tumors were extracted from 4 to 6 mice randomly selected from each therapy group. The removed tumor was fixed with 10 v/v % neutral formalin. By requesting an experimental animal pathology platform of the Tohoku University, paraffin-embedded tissue sections were prepared from the fixed tumor, and immunostaining was performed using rabbit anti-Ki-67 antibodies (Cell Signaling Technology, dilution ratio: 1:800). The stained sample was observed with an optical microscope at a magnification of 400×. Five fields of view for a central part of the tumor without tumor necrosis were selected, and 500 or more cells in total were counted in each group. The Ki-67 labeling index was calculated as the number of Ki-67 positive cells/the total number of cells.

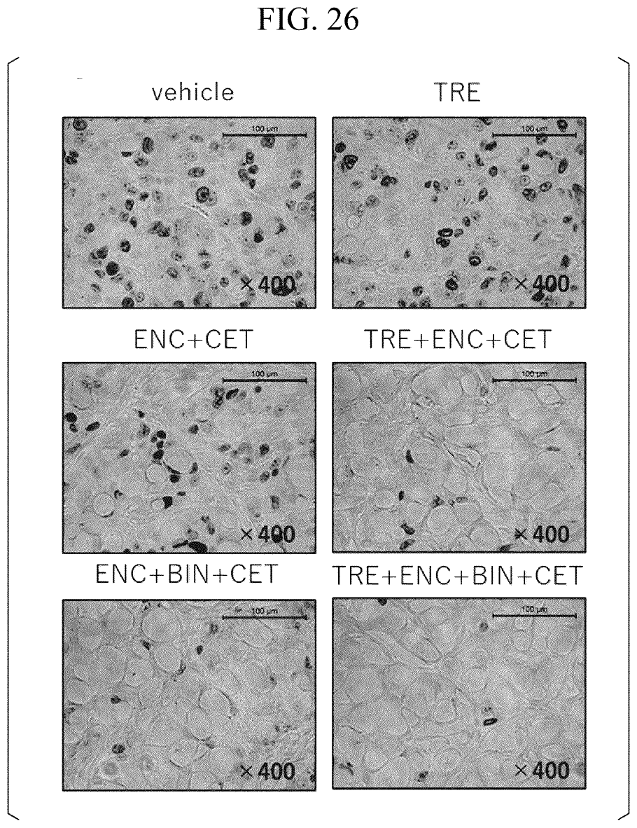

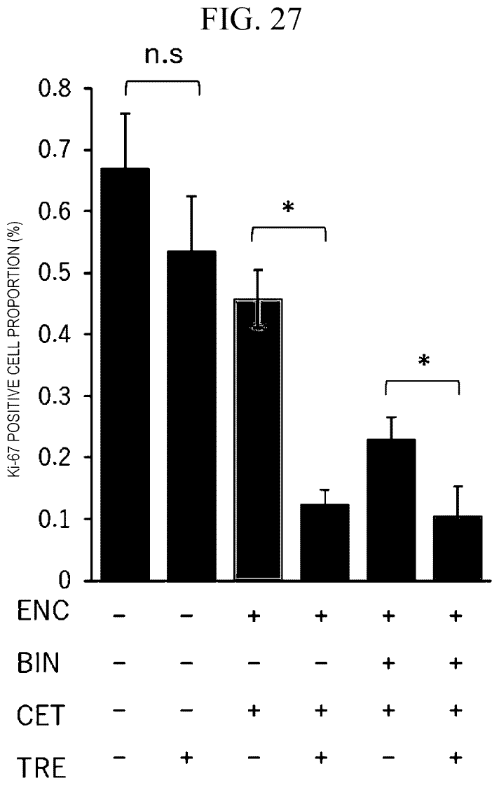

FIG. 26 shows immunohistochemical staining images (magnification: 400×), using anti-Ki-67 antibodies, of the subcutaneous transplanted tumor mouse model. In FIG. 26, the scale bar is 100 μm. FIG. 27 shows a Ki-67 positive cell proportion (%) calculated using the stained image in FIG. 26.

No significant difference was recognized between the Ki-67 labeling indexes of the vehicle group and the TRE single group. On the other hand, the Ki-67 labeling indexes of the TRE+ENC+CET group and the TRE+ENC+BIN+CET group were significantly lower than those of the ENC+CET group and the ENC+BIN+CET group, respectively.

From these results, it has been shown in the in vivo model that TRE synergistically enhances antitumor effects when added to ENC+CET or ENC+BIN+CET, and these therapies are well tolerated.

From the above results, it has been found that a compound acting on a retinoid receptor, such as TRE, RET, TAM, or BEX, is a compound that enhances the apoptosis inducing ability mediated through RARα or RXRα by a BRAF inhibitor and an MEK inhibitor, and synergistically enhances the effect of inhibiting proliferation of BRAF-mutated colorectal cancer cells. Further, it has been revealed for the first time that a compound acting on a retinoid receptor, such as TRE, enhances the antitumor effect of a BRAF inhibitor, an MEK inhibitor, and an anti-EGFR antibody even in an in vivo model. This test has shown the possibility that a compound acting on a retinoid receptor, such as TRE, becomes a promising novel therapeutic agent for a BRAF-mutated colorectal cancer.

### INDUSTRIAL APPLICABILITY

According to the therapeutic agent for cancer of the invention, it is possible to provide a therapeutic agent for cancer, which contains a component exhibiting a synergistic effect with a BRAF inhibitor, exhibits a strong antitumor effect, exhibits an effect of enhancing a therapeutic effect on cells resistive to a BRAF inhibitor, an anti-EGFR antibody, and an MEK inhibitor, and is particularly effective for therapy for a gene-mutated cancer.

1. A therapeutic agent for cancer, comprising:
a compound acting on a retinoid receptor; and
a BRAF inhibitor.

2. A therapeutic agent for cancer to be used in combination with a BRAF inhibitor, the therapeutic agent comprising:
a compound acting on a retinoid receptor.

3. The therapeutic agent for cancer according to claim 1, wherein
the compound acting on a retinoid receptor is a retinoid compound or a derivative thereof.

4. The therapeutic agent for cancer according to claim 1, wherein
the compound acting on a retinoid receptor is tretinoin, tamibarotene, or bexarotene.

5. The therapeutic agent for cancer according to claim 1, wherein
the BRAF inhibitor is dabrafenib or encorafenib.

6. The therapeutic agent for cancer according to claim 1, further comprising:
an MEK inhibitor.

7. The therapeutic agent for cancer according to claim 6, wherein
the MEK inhibitor is trametinib or binimetinib.

8. The therapeutic agent for cancer according to claim 1, further comprising:
a compound acting on an epidermal growth factor or an epidermal growth factor receptor.

9. The therapeutic agent for cancer according to claim 8, wherein
the compound acting on an epidermal growth factor or an epidermal growth factor receptor is bevacizumab, cetuximab, or panitumumab.

10. The therapeutic agent for cancer according to claim 1, which is a therapeutic agent for a BRAF-mutated cancer.

11. The therapeutic agent for cancer according to claim 1, which is a therapeutic agent for a colorectal cancer.
